SEQUENCE LISTING (9 sequences)

SEQ ID NO: 1 - DNA, 132 bp, Homo sapiens
aaggcagccaacatccctctggtgtcagaacttgccatcgatgacattcattttgatgacttttctctcgacgttgatgccatgatcacagctgctctccggatgttcatggagctggggatggtacagaaa

SEQ ID NO: 2 - DNA, 132 bp, Homo sapiens
ttccgtcggttgtagggagaccacagtcttgaacggtagctactgtaagtaaaactactgaaaagagagctgcaactacggtactagtgtcgacgagaggcctacaagtacctcgacccctaccatgtcttt

SEQ ID NO: 3 - DNA, 25 bp, Homo sapiens
tttctgtaccatccccagctccatg

SEQ ID NO: 4 - DNA, 25 bp, Homo sapiens
aaggcagccaacatccctctggtgt

SEQ ID NO: 5 - DNA, 83 bp, Mus musculus
tcggaactggccatcgatgacatccatttcgatgacttttcccttgatgttgatgccatgatcacagccgctctacggatgtt

SEQ ID NO: 6 - DNA, 83 bp, Mus musculus
agccttgaccggtagctactgtaggtaaagctactgaaaagggaactacaactacggtactagtgtcggcgagatgcctacaa

SEQ ID NO: 7 - DNA, 10 bp, Mus musculus
atgacattca

SEQ ID NO: 8 - DNA, 10 bp, Mus musculus
ctcgacgttg

SEQ ID NO: 9 - protein, 15 aa, Homo sapiens
SAIFDRNRKDELPRL

(19) United States
(12) Patent Application Publication
(10) Pub. No.: US 2003/0229002 A1
(43) Pub. Date: Dec. 11, 2003

(54) Use of agents that modulate PDE11A activity

(75) Inventors: Martyn Frank Burslem, Sandwich (GB); Ian Dennis Harrow, Sandwich (GB); Jeremy Lanfear, Sandwich (GB); Stephen Charles Phillips, Sandwich (GB); Christopher Peter Wayman, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/366,923

(22) Filed: Feb. 14, 2003

(30) Foreign Application Priority Data
Feb. 22, 2002 (GB) 0204227.3

(51) Int. Cl. A61K 31/00; A61K 31/519

(52) U.S. Cl. 514/1; 514/252.16

(57) ABSTRACT

The present invention relates to the effect of PDE11A modulation on male pro-fertility, male contraception, and female sexual dysfunction (FSD), specifically female sexual arousal disorder (FSAD), female orgasmic disorder (FOD), hypoactive sexual desire disorder (HSDD) or sexual pain disorders.

### FIELD OF THE INVENTION

The present invention relates to the effect of PDE11A modulation (i.e. PDE11A inhibition or stimulation) on in vivo or ex vivo spermatozoa capacitation and PDE11A stimulation on in vivo spermatozoa capacitation. The invention also relates to the effect of PDE11A modulation on male pro-fertility, male contraception, and female sexual dysfunction (FSD), specifically female sexual arousal disorder (FSAD), female orgasmic disorder (FOD), hypoactive sexual desire disorder (HSDD) or sexual pain disorders.

### BACKGROUND OF THE INVENTION

Cyclic nucleotide phosphodiesterases (PDEs) catalyze the hydrolysis of cyclic nucleotides, such as the second messengers cAMP (cyclic adenosine 3′,5′-monophosphate) and cGMP (cyclic guanine 3′,5′-monophosphate). Thus, PDEs play a pivotal regulatory role in a wide variety of signal transduction pathways (Beavo, Physiol. Rev. 75: 725-48, 1995). For example, PDEs mediate processes involved in vision (McLaughlin et al., Nat. Genet. 4: 130-34, 1993), olfaction (Yan et al., Proc. Natl. Acad. Sci. USA 92: 9677-81, 1995), platelet aggregation (Dickinson et al. Biochem. J. 323: 371-77, 1997), aldosterone synthesis (MacFarland et al., J. Biol. Chem. 266: 136-42, 1991), insulin secretion (Zhao et al., J. Clin. Invest. 102: 869-73, 1998), T-cell activation (Li et al., Science 283: 848-51, 1999), and smooth muscle relaxation (Boolell et al., Int. J. Impot. Res. 8: 47-52, 1996; Ballard et al., J. Urol. 159: 2164-71, 1998).

PDEs form a superfamily of enzymes that are subdivided into 11 major families (Beavo, Physiol. Rev. 75: 725-48, 1995; Beavo et al., Mol. Pharmacol. 46: 399-05, 1994; Soderling et al., Proc. Natl. Acad. Sci. USA 95: 8991-96, 1998; Fisher et al., Biochem. Biophys. Res. Commun. 246: 570-77, 1998; Hayashi et al., Biochem. Biophys. Res. Commun. 250: 751-56, 1998; Soderling et al., J. Biol. Chem. 273: 15553-58, 1998; Fisher et al., J. Biol. Chem. 273: 15559-64, 1998; Soderling et al., Proc. Natl. Acad. Sci. USA 96: 7071-76, 1999; and Fawcett et al., Proc. Natl. Acad. Sci. USA 97: 3702-07, 2000).

Each PDE family is distinguished functionally by unique enzymatic characteristics and pharmacological profiles. In addition, each family exhibits distinct tissue, cell, and subcellular expression patterns (Beavo et al., Mol. Pharmacol. 46: 399-405, 1994; Soderling et al., Proc. Natl. Acad. Sci. USA 95: 8991-96, 1998; Fisher et al., Biochem. Biophys. Res. Commun. 246: 570-77, 1998; Hayashi et al., Biochem. Biophys. Res. Commun. 250: 751-56, 1998; Soderling et al., J. Biol. Chem. 273: 15553-58, 1998; Fisher et al., J. Biol. Chem. 273: 15559-64, 1998; Soderling et al., Proc. Natl. Acad. Sci. USA 96: 7071-76, 1999; Fawcett et al., Proc. Natl. Acad. Sci. USA 97: 3702-07, 2000; Boolell et al., Int. J. Impot. Res. 8: 47-52, 1996; Ballard et al., J. Urol. 159: 2164-71, 1998; Houslay, Semin. Cell Dev. Biol. 9: 161-67, 1998; and Torphy et al., Pulm. Pharmacol. Ther. 12: 131-35, 1999). Therefore, by administering a compound that selectively regulates the activity of one family or subfamily of PDE enzymes, it is possible to regulate cAMP and/or cGMP signal transduction pathways in a cell- or tissue-specific manner.

Ren et al. (Nature, Vol. 413: 603-609, 2001) provides a link between cAMP elevation and Ca²⁺ influx in spermatozoa (via a newly discovered, testis-specific, cyclic nucleotide monophosphate (cNMP)-gated calcium channel—sperm cation channel, CatSper), which induces capacitation and dowstream events leading to fertilisation capability. Knock-out (KO) of the channel (CatSper −/− mice) leads to normal looking testis and spermatozoa, but the male mice are infertile due to an inability to ‘activate’ the spermatozoa.

PDE11 is one of the most recently described families of PDEs; PDE11A is the sole member of this family so far identified (Fawcett et al., Proc. Natl. Acad. Sci. USA 97: 3702-07, 2000, hereinafter “Fawcett, 2000,38 Yuasa et al., J. Biol. Chem. 275: 31469-79, 2000, hereinafter “Yuasa, 2000”). While PDE11A is known to be expressed in, e.g., testis, skeletal muscle, kidney, liver, various glandular tissue (e.g., pituitary, salivary, adrenal, mammary, and thyroid), pancreas, spinal cord, and trachea (Fawcett, 2000), little is known about PDE11A function.

Human PDE11A exhibits >50% amino acid identity with the catalytic domains of all other mammalian PDEs, being most similar to human PDE5, and has distinct biochemical properties; this family now comprises 4 isoforms, i.e., PDE11A1-4 (Hetman J M, Robas N, Baxendale R, Fidock M, Phillips S C, Soderling S H, Beavo J A. Cloning and characterization of two splice variants of human phosphodiesterase 11A. (Proc Natl Acad Sci USA. 2000; 97(23): 12891-5); Yuasa, 2000). Recombinant human PDE11A hydrolyses both cGMP and cAMP with Kₘ values of 0.52 μM and 1.04 μM respectively and similar Vₘₐₓ values. Therefore, PDE11A represents a dual-substrate PDE that may regulate both cGMP and cAMP under physiological conditions.

Patent applications CA 2,360,485 (filed Oct. 30, 2001), EP 01308959.4 (filed Oct. 22, 2001), JP 2001-337061 (filed Nov. 1, 2001) and US (no serial number available—not yet issued by USPTO) disclose biological tools (e.g. genetically-modified non-human mammals and genetically-modified animal cells containing a functionally disrupted PDE11A gene) to study PDE11A function and methods to identify agents (e.g. methods of screening for agents that modulate PDE11A and methods of modulating cAMP and cGMP signal transduction in cells that express PDE11A) that regulate PDE11A activity for use in preventing or treating diseases and conditions that are linked to these PDE11A functions.

Patent applications CA 2,360,485 (filed Oct. 30, 2001), EP 01308959.4 (filed Oct. 22, 2001), JP 2001-337061 (filed Nov. 1, 2001) and US (no serial number available—not yet issued by USPTO) disclose the provision of a PDE11A knockout mouse, which provides an excellent opportunity to investigate genes involved in, inter alia, spermatogenesis, and, when compared with the wild type mouse, to dissect out the components involved in spermatogenesis. One method for this type of analysis is microarray technology. With DNA microarray technology, it becomes possible to monitor large-scale gene expression over time. Prefabricated arrays of large numbers of especially designed oligonucleotide probes, e.g. as manufactured by Affymetrix (CA, USA), enable simultaneous hybridization-based analysis of thousands of genes.

The findings of Ren et al. (see above) link well with the findings described herein, in that knockout (KO) of PDE11A (PDE11A −/− mice) results in elevated cAMP/cGMP levels, which in turn activate downstream signalling (Ca²⁺ influx via the above channel—Cat Sper) and hence premature capacitation/‘activation’.

### SUMMARY OF THE INVENTION

The present invention provides the following (numbered) aspects:

1. A method of modulating in vivo or ex vivo spermatozoa capacitation, said method comprising administering an agent that modulates PDE11A activity.

The term “in vivo spermatozoa capacitation” refers to the capacitation of spermatozoa whilst within the testis (i.e. non-ejaculated spermatozoa). Capacitation results in spermatozoa becoming “switched on” and able to undergo the acrosome reaction and ultimately to fertilize an oocyte. However, when such capacitation of spermatozoa occurs in vivo, then the spermatozoa that are eventually ejaculated are, for the most part, less likely to survive the journey to the oocyte and the site of fertilization (essentially the spermatozoa have been prematurely capacitated). This leads to in vivo spermatozoa capacitation being a form of male contraception. It should be noted, however, that if the modulation of in vivo spermatozoa capacitation results in a reduction in capacitation, then male pro-fertility may result. This is because fewer spermatozoa will have been prematurely “switched on” in the testis and so the majority of spermatozoa will be able to undergo (later) the acrosome reaction and ultimately to fertilize an oocyte (after having survived (better) the journey to the oocyte and the site of fertilization).

The term “ex vivo spermatozoa capacitation” refers to the capacitation of spermatozoa when outside the male body (i.e. ejaculated spermatozoa or spermatozoa removed from the body artificially). Ex vivo is, in this context, synonymous with in vitro. Ex vivo or in vitro spermatozoa capacitation is of primary use for ensuring that the majority of spermatozoa that may be used for fertilizing an oocyte during an in vitro fertilization (IVF) procedure (or similar pro-fertility technique/treatment) are “switched on” and able to undergo the acrosome reaction and ultimately to fertilize an oocyte. This thus leads to ex vivo or in vitro spermatozoa capacitation being a male pro-fertility mechanism. It should be noted, however, that if the modulation of ex vivo spermatozoa capacitation results in a reduction in capacitation (which would not be desirable ex vivo), then male contraception may result. This is because fewer spermatozoa will have been “switched on” prior to use in an in vitro fertilization (IVF) procedure and so the majority of spermatozoa may not be able to undergo the acrosome reaction and ultimately to fertilize an oocyte.

2. The method according to aspect 1, wherein said agent reduces PDE11A activity and increases in vivo or ex vivo spermatozoa capacitation.

3. The method according to aspect 1 or aspect 2, wherein said agent is (a) a PDE11A inhibitor or antagonist or (b) an inhibitor of PDE11A gene expression.

As used herein, the terms PDE11A inhibitor or PDE11A antagonist refer to any compound or agent which is able to inhibit or reduce the activity of PDE11A (or reduce the levels of PDE11A) or which is able to decrease the level of gene expression of PDE11A.

4. The method according to any one of aspects 1, 2 or 3(a), wherein said agent is Cialis (IC351), E4021 or UK-235,187.

Details of the above-mentioned agents:

Cialis (IC351; ICOS, Bothell, USA)

Patent Application: WO 95/19978 (Glaxo)

CAS number: 171488-01-0

Name: (6R,12aR)-6-(1,3-benzodioxol-5-yl)-2,3,6,7,12,12a-hexahydro-2-methyl-pyrazino[1′,2′: 1,6]pyrido[3,4-b]indole-1,4-dione

Chemical structure:

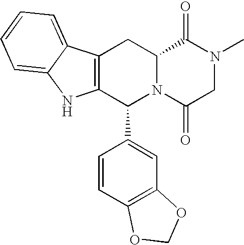

E4021 (Eisai, Ibaraki, Japan)

Patent Application: WO 93/07124 (Eisai)

CAS number: 150452-21-4

Name: 1-[4-[(1,3-benzodioxol-5-ylmethyl)amino]-6-chloro-2-quinazolinyl]-4-piperidinecarboxylic acid, monohydrochloride

Chemical structure:

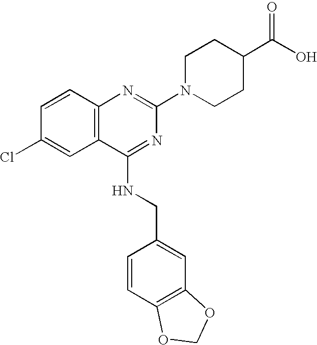

UK-235,187 (Internal Compound Code for representative compound from series described by Ono Pharmaceuticals, Osaka, Japan)

Patent Application: EP 579496

CAS number: 157863-31-5

Name: 6-Chloro-2-(1H-imidazol-1-yl)-N-(phenylmethyl)-,4-quinazolinamine, dihydrochloride

Chemical structure:

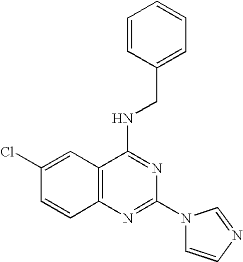

5. The method according to aspect 1, wherein said agent increases PDE11A activity and decreases in vivo or ex vivo spermatozoa capacitation.

6. The method according to aspect 1 or aspect 5, wherein said agent is (a) a PDE11A stimulator, activator or agonist or (b) a stimulator, enhancer or activator of PDE11A gene expression.

As used herein, the terms PDE11A stimulator, PDE11A activator, PDE11A enhancer or PDE11A agonist refer to any compound or agent which is able to maximise or increase the activity of PDE11A (or increase the levels of PDE11A) or which is able to increase the level of gene expression of PDE11A.

7. Use of an agent that modulates PDE11A activity in the manufacture of a medicament for modulating in vivo or ex vivo spermatozoa capacitation.

8. Use according to aspect 7, wherein said agent reduces PDE11A activity and increases in vivo or ex vivo spermatozoa capacitation.

9. Use according to aspect 7 or aspect 8, wherein said agent is (a) a PDE11A inhibitor or antagonist or (b) an inhibitor of PDE11A gene expression.

10. Use according to any one of aspects 7, 8 or 9(a), wherein said agent is Cialis (IC351), E4021 or UK-235,187.

11. Use of an agent that increases PDE11A activity in the manufacture of a medicament for decreasing in vivo or ex vivo spermatozoa capacitation.

12. Use according to aspect 7 or aspect 11, wherein said agent is (a) a PDE11A stimulator, activator or agonist or (b) a stimulator, enhancer or activator of PDE11A gene expression.

13. Use of an agent that reduces PDE11A activity as a male pro-fertility agent, wherein said male pro-fertility agent is used ex vivo.

14. Use according to aspect 13, wherein said male pro-fertility agent is (a) a PDE11 A inhibitor or antagonist or (b) an inhibitor of PDE11A gene expression.

15. Use according to aspect 13 or aspect 14(a), wherein said agent that reduces PDE11A activity is Cialis (IC351), E4021 or UK-235,187.

16. Use of an agent that increases PDE11A activity as a male pro-fertility agent, wherein said male pro-fertility agent is used in vivo.

17. Use according to aspect 16, wherein said male pro-fertility agent is (a) a PDE11A stimulator, activator or agonist or (b) a stimulator, enhancer or activator of PDE11A gene expression.

18. Use of an agent that increases spermatozoa capacitation as a male pro-fertility agent, wherein said male pro-fertility agent is used ex vivo.

19. Use according to aspect 18, wherein said male pro-fertility agent is (a) a PDE11A inhibitor or antagonist or (b) an inhibitor of PDE11A gene expression.

20. Use according to aspect 18 or aspect 19(a), wherein said agent that increases spermatozoa capacitation is Cialis (IC351), E4021 or UK-235,187.

21. Use of an agent that reduces spermatozoa capacitation as a male pro-fertility agent, wherein said male pro-fertility agent is used in vivo.

22. Use according to aspect 21, wherein said male pro-fertility agent is (a) a PDE11A stimulator, activator or agonist or (b) a stimulator, enhancer or activator of PDE11A gene expression.

23. Use of an agent that reduces PDE11A activity as a male contraceptive agent, wherein said male contraceptive agent is used in vivo.

24. Use according to aspect 23, wherein said male contraceptive agent is (a) a PDE11A inhibitor or antagonist or (b) an inhibitor of PDE11A gene expression.

25. Use according to aspect 23 or aspect 24(a), wherein said agent that reduces PDE11A activity is Cialis (IC351), E4021 or UK-235,187.

26. Use of an agent that increases PDE11A activity as a male contraceptive agent, wherein said male contraceptive agent is used ex vivo.

27. Use according to aspect 26, wherein said male contraceptive agent is (a) a PDE11A stimulator, activator or agonist or (b) a stimulator, enhancer or activator of PDE11A gene expression.

28. Use of an agent that increases spermatozoa capacitation as a male contraceptive agent, wherein said male contraceptive agent is used in vivo.

29. Use according to aspect 28, wherein said male contraceptive agent is (a) a PDE11A inhibitor or antagonist or (b) an inhibitor of PDE11A gene expression.

30. Use according to aspect 28 or aspect 29(a), wherein said agent that increases spermatozoa capacitation is Cialis (IC351), E4021 or UK-235,187.

31. Use of an agent that reduces spermatozoa capacitation as a male contraceptive agent, wherein said male contraceptive agent is used ex vivo.

32. Use according to aspect 31, wherein said male contraceptive agent is (a) a PDE11A stimulator, activator or agonist or (b) a stimulator, enhancer or activator of PDE11A gene expression.

33. A method of preventing or treating female sexual dysfunction (FSD), said method comprising administering to a female mammal an agent that stimulates, activates, enhances or agonises PDE11A activity.

34. A method according to aspect 33, wherein said female sexual dysfunction (FSD) is female sexual arousal disorder (FSAD), female orgasmic disorder (FOD) or a sexual pain disorder. Preferably said sexual pain disorder is dyspareunia or vaginismus.

35. A method according to aspect 33 or aspect 34, wherein said female mammal is a human female.

36. Use of an agent that stimulates, activates, enhances or agonises PDE11A activity in the manufacture of a medicament for the prevention or treatment of female sexual dysfunction (FSD).

37. Use according to aspect 36, wherein said female sexual dysfunction (FSD) is female sexual arousal disorder (FSAD), female orgasmic disorder (FOD) or a sexual pain disorder. Preferably said sexual pain disorder is dyspareunia or vaginismus.

38. Use according to aspect 36 or aspect 37, wherein said female mammal is a human female.

39. A method of preventing or treating female sexual dysfunction (FSD), said method comprising administering to a female mammal an agent that inhibits, decreases, deactivates or antagonises PDE11A activity.

40. A method according to aspect 39, wherein said female sexual dysfunction (FSD) is hypoactive sexual desire disorder (HSDD).

41. A method according to aspect 39 or aspect 40, wherein said female mammal is a human female.

42. The method according to any one of aspects 39 to 41, wherein said agent that inhibits, decreases, deactivates or antagonises PDE11A activity is Cialis (IC351), E4021 or UK-235,187.

43. Use of an agent that inhibits, decreases, deactivates or antagonises PDE11A activity in the manufacture of a medicament for the prevention or treatment of female sexual dysfunction (FSD).

44. Use according to aspect 42, wherein said female sexual dysfunction (FSD) is hypoactive sexual desire disorder (HSDD).

45. Use according to aspect 42 or aspect 43, wherein said female mammal is a human female.

46. Use according to any one of aspects 43 to 45, wherein said agent that inhibits, decreases, deactivates or antagonises PDE11A activity is Cialis (IC351), E4021 or UK-235,187.

Modulation of PDE11A activity has a modulating effect on certain other proteins, for example: Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen, HR6A, Protamine 1, sp32, mCDC46, Adenylate kinase 2, AKAP121 or Krox-24 binding protein. More specifically, an increase in PDE11A activity increases the levels of e.g. Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen and HR6A, but decreases the levels of e.g. Protamine 1, sp32, mCDC46, Adenylate kinase 2, AKAP121 and Krox-24 binding protein. Conversely, a decrease in PDE11A activity decreases the levels of e.g. Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen and HR6A, but increases the levels of e.g. Protamine 1, sp32, mCDC46, Adenylate kinase 2, AKAP121 and Krox-24 binding protein.

Thus, by modulating, for example, Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen, HR6A, Protamine 1, sp32, mCDC46, Adenylate kinase 2, AKAP121 or Krox-24 binding protein, one can modulate or affect spermatozoa capacitation, sexual desire (libido), sexual arousal or orgasm.

Preferably, the modulating agent of the present invention reduces the levels of Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen or HR6A, thereby increasing spermatozoa capacitation in vivo or ex vivo, increasing sexual desire (libido), decreasing sexual arousal or decreasing orgasm. More preferably, said agent is (a) an inhibitor or antagonist of Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen or HR6A, or (b) an inhibitor of the gene expression of Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen or HR6A.

Inhibitor or antagonist refers to any compound or agent which is able to inhibit or reduce the activity (or level) of any of the above proteins or decrease their level of expression.

Most preferably, said agent is Cialis (IC351), E4021 or UK-235,187.

In the alternative, the modulating agent of the present invention increases the levels of Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen or HR6A, thereby reducing spermatozoa capacitation in vivo or ex vivo, decreasing sexual desire (libido), increasing sexual arousal or increasing orgasm. More preferably, said agent is (a) a stimulator, activator or agonist of Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen or HR6A, or (b) a stimulator, enhancer or activator of the gene expression of Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen or HR6A.

Stimulator, activator, enhancer or agonist refers to any compound or agent which is able to maximise or increase the activity (or level) of any of the above proteins or increase their level of expression.

In a further embodiment of the present invention, the modulating agent of the present invention reduces the levels of Protamine 1, sp32, mCDC46, Adenylate kinase 2, AKAP121 or Krox-24 binding protein, thereby reducing spermatozoa capacitation in vivo or ex vivo, decreasing sexual desire (libido), increasing sexual arousal or increasing orgasm. Preferably, said agent is (a) an inhibitor or antagonist of Protamine 1, sp32, mCDC46, Adenylate kinase 2, AKAP121 or Krox-24 binding protein or (b) an inhibitor of the gene expression of Protamine 1, sp32, mCDC46, Adenylate kinase 2, AKAP121 or Krox-24 binding protein.

Inhibitor or antagonist refers to any compound or agent which is able to inhibit or reduce the activity (or level) of any of the above proteins or decrease their level of expression.

In yet a further alternative embodiment, the modulating agent of the present invention increases the levels of Protamine 1, sp32, mCDC46, Adenylate kinase 2, AKAP121 or Krox-24 binding protein, thereby increasing spermatozoa capacitation in vivo or ex vivo, increasing sexual desire (libido), decreasing sexual arousal or decreasing orgasm. Preferably, said agent is (a) a stimulator, activator or agonist of Protamine 1, sp32, mCDC46, Adenylate kinase 2, AKAP121 or Krox-24 binding protein or (b) a stimulator, enhancer or activator of the gene expression of Protamine 1, sp32, mCDC46, Adenylate kinase 2, AKAP121 or Krox-24 binding protein.

Stimulator, activator, enhancer or agonist refers to any compound or agent which is able to maximise or increase the activity (or level) of any of the above proteins or increase their level of expression.

More preferably, said agent is Cialis (IC351), E4021 or UK-235,187.

For the avoidance of doubt, the present invention teaches that an agent that reduces PDE11A levels/activity can also reduce the levels/activity of Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen or HR6A, but increase the levels/activity of Protamine 1, sp32, mCDC46, Adenylate kinase 2, AKAP121 or Krox-24 binding protein. Conversely, an agent that increases PDE11A levels/activity can also increase the levels/activity of Corticosteroid binding globulin, Centrin 3, XRCC1, Chromobox M33, GABA-A (gamma 3 sub-unit), Prohormone convertase 5, Leydig Insulin-like peptide, Calpain 3, Y-Box 3, Chromogranin B, Cryptdin I, PP2B, Glutamate cysteine ligase, Nidogen or HR6A, but decrease the levels/activity of Protamine 1, sp32, mCDC46, Adenylate kinase 2, AKAP121 or Krox-24 binding protein.

Also for the avoidance of doubt, the present invention teaches that if sexual desire (libido) in female mammals, preferably human females, is increased, then the female sexual dysfunction (FSD) known as hypoactive sexual desire disorder (HSDD) can be treated. Furthermore, if sexual arousal in female mammals, preferably human females, is increased, then the FSD known as female sexual arousal disorder (FSAD) can be treated. Yet further, if orgasm in female mammals, preferably human females, is increased, then the FSD known as female orgasmic disorder (FOD) or female sexual orgasmic disorder (FSOD) can be treated. Further still, if a female mammal, preferably a human female, is sexually aroused, then the lubrication-swelling response of sexual excitement will help to treat the FSD group known as sexual pain disorders, such as dyspareunia and vaginismus.

It will be appreciated that the present invention provides that the modulation of the PDE11A activity can be either a reduction in PDE11A activity/levels, leading to, inter alia, an increase in spermatozoa capacitation, or an increase in PDE11A activity/levels, leading to, inter alia, a decrease in spermatozoa capacitation (see FIGS. 7A and 7B).

Agents of the invention that result in an increase in spermatozoa capacitation can be classed as male in vivo contraceptive agents or male ex vivo pro-fertility agents (see FIG. 7A).

Agents of the invention that result in a decrease in spermatozoa capacitation can be classed as male in vivo pro-fertility agents or male ex vivo contraceptive agents (see FIG. 7B).

Those skilled in the art will fully understand the terms used herein in the description and the appendant claims to describe the present invention. Nonetheless, unless otherwise provided herein, the following terms are as described immediately below.

By “inhibitor” or “antagonist” is meant any molecule/compound/agent that is capable of eliciting a decrease in activity/production levels of the target object (e.g. PDE11A enzyme or PDE11A gene) of the inhibitor or antagonist.

By “stimulator”, “activator”, “enhancer” or “agonist” is meant any molecule/compound/agent that is capable of eliciting an increase in activity/production levels of the target object (e.g. PDE11A enzyme or PDE11A gene) of the stimulator, activator, enhancer or agonist.

By “modulator” is meant an “inhibitor”, “antagonist”, “stimulator”, “activator”, “enhancer” or “agonist” (i.e. any molecule/compound/agent that modulates the activity/production levels of the target object (e.g. PDE11A enzyme or PDE11A gene) of the inhibitor, antagonist, stimulator, activator, enhancer or agonist).

By “coding sequence” or “coding region” is meant a nucleic acid molecule having sequence information necessary to produce a gene product when the sequence is expressed.

By “antisense oligonucleotide” is meant a small nucleic acid molecule, typically between about 10 to 50 nucleotides long, which may be unmodified RNA or DNA or modified RNA or DNA; it is designed to hybridize to the respective transcript, and thereby reduce its translation into protein, e.g. by blocking translation or by facilitating rapid degradation of the respective transcript.

By “ribozyme” is meant a nucleic acid molecule, which can be unmodified RNA or DNA or modified RNA or DNA, which is designed to hybridize and cleave the respective transcript.

The term “antibody” as used herein includes polyclonal and monoclonal antibodies, single chain, chimeric and humanized antibodies, as well as antibody fragments, whether produced by recombinant or proteolytic means. The term is also meant to include the products of any antibody-derived expression libraries, e.g. single-chain Fv or Fab fragment expression libraries.

By “biomarker/marker” is meant any molecule, e.g. a transcript of the gene or the translation product, which is indicative of a condition, disorder, disease, or the status in the progression of a process, e.g. spermatogenesis, or the status of the progression of a disease.

A non-human mammal or an animal cell that is “genetically-modified” is heterozygous or homozygous for a modification that is introduced into the non-human mammal or animal cell, or into a progenitor non-human mammal or animal cell, by genetic engineering. The standard methods of genetic engineering that are available for introducing the modification include homologous recombination, viral vector gene trapping, irradiation, chemical mutagenesis, and the transgenic expression of a nucleotide sequence encoding antisense RNA alone or in combination with catalytic ribozymes. Preferred methods for genetic modification are those which modify an endogenous gene by inserting a “foreign nucleic acid sequence” into the gene locus, e.g., by homologous recombination or viral vector gene trapping. A “foreign nucleic acid sequence” is an exogenous sequence that is non-naturally occurring in the gene. This insertion of foreign DNA can occur within any region of the PDE11A gene, e.g., in an enhancer, promoter, regulator region, noncoding region, coding region, intron, or exon. The most preferred method of genetic engineering is homologous recombination, in which the foreign nucleic acid sequence is inserted in a targeted manner either alone or in combination with a deletion of a portion of the endogenous gene sequence.

By a PDE11A gene that is “functionally disrupted” is meant a PDE11A gene that is genetically modified such that the cellular activity of the PDE11A polypeptide encoded by the disrupted gene is decreased in cells that normally express a wild type version of the PDE11A gene. When the genetic modification effectively eliminates all wild type copies of the PDE11A gene in a cell (e.g., the genetically-modified, non-human mammal or animal cell is homozygous for the PDE11A gene disruption or the only wild type copy of PDE11A gene originally present is now disrupted), then the genetic modification results in a reduction in PDE11A polypeptide activity as compared to an appropriate control cell that expresses the wild type PDE11A gene. This reduction in PDE11A polypeptide activity results from either reduced PDE11A gene expression (i.e., PDE11A mRNA levels are effectively reduced and produce reduced levels of PDE11A polypeptide) and/or because the disrupted PDE11A gene encodes a mutated polypeptide with reduced function or stability as compared to a wild type PDE11A polypeptide. Preferably, the activity of PDE11A polypeptide in the genetically-modified, non-human mammal or animal cell is reduced to 50% or less of wild type levels, more preferably, to 25% or less, and, even more preferably, to 10% or less of wild type levels. Most preferably, the PDE11A gene disruption results in non-detectable PDE11A activity.

By a “genetically-modified, non-human mammal” containing a functionally disrupted PDE11A gene is meant a non-human mammal that is originally produced, for example, by creating a blastocyst or embryo carrying the desired genetic modification and then implanting the blastocyst or embryo in a foster mother for in utero development. The genetically-modified blastocyst or embryo can be made, in the case of mice, by implanting a genetically-modified embryonic stem (ES) cell into a mouse blastocyst or by aggregating ES cells with tetraploid embryos. Alternatively, various species of genetically-modified embryos can be obtained by nuclear transfer. In the case of nuclear transfer, the donor cell is a somatic cell or a pluripotent stem cell, and it is engineered to contain the desired genetic modification that functionally disrupts the PDE11A gene. The nucleus of this cell is then transferred into a fertilized or parthenogenetic oocyte that is enucleated; the embryo is reconstituted, and developed into a blastocyst. A genetically-modified blastocyst produced by either of the above methods is then implanted into a foster mother according to standard methods well known to those skilled in the art. A “genetically-modified, non-human mammal” includes all progeny of the mammals created by the methods described above, provided that the progeny inherit at least one copy of the genetic modification that functionally disrupts the PDE11A gene. It is preferred that all somatic cells and germline cells of the genetically-modified mammal contain the modification. Preferred non-human animals that are genetically-modified to contain a disrupted PDE11A gene include rodents, such as mice and rats, cats, dogs, rabbits, guinea pigs, hamsters, sheep, pigs, and ferrets.

By a “genetically-modified animal cell” containing a functionally disrupted PDE11A gene is meant an animal cell, including a human cell, created by genetic engineering to contain a functionally disrupted PDE11A gene, as well as daughter cells that inherit the disrupted PDE11A gene. These cells may be genetically-modified in culture according to any standard method known in the art. As an alternative to genetically modifying the cells in culture, non-human mammalian cells may also be isolated from a genetically-modified, non-human mammal that contains a PDE11A gene disruption. The animal cells of the invention may be obtained from primary cell or tissue preparations as well as culture-adapted, tumorigenic, or transformed cell lines. These cells and cell lines are derived, for example, from endothelial cells, epithelial cells, islets, neurons and other neural tissue-derived cells, mesothelial cells, osteocytes, lymphocytes, chondrocytes, hematopoietic cells, immune cells, cells of the major glands or organs (e.g., testicle, liver, lung, heart, stomach, pancreas, kidney, and skin), muscle cells (including cells from skeletal muscle, smooth muscle, and cardiac muscle), exocrine or endocrine cells, fibroblasts, and embryonic and other totipotent or pluripotent stem cells (e.g., ES cells, ES-like cells, and embryonic germline (EG) cells, and other stem cells, such as progenitor cells and tissue-derived stem cells). The preferred genetically-modified cells are ES cells, more preferably, mouse or rat ES cells, and, most preferably, human ES cells.

By an “ES cell” or an “ES-like cell” is meant a pluripotent stem cell derived from an embryo, from a primordial germ cell, or from a teratocarcinoma, that is capable of indefinite self-renewal as well as differentiation into cell types that are representative of all three embryonic germ layers.

By a “PDE11A gene” is meant the nucleic acid sequences encoding the polypeptides disclosed in Fawcett, 2000, (human PDE11A1, GenBank Accession No. AJ251509), WO 00/40733 (human PDE11A1 and PDE11A2), or Yuasa, 2000 (human PDE11A3 and PE11A4, GenBank Accession Nos. AB036704 and AB038041), as well as any human allelic variants and any mammalian sequences encoding homologues having PDE11A activity and their allelic variants. By a “PDE11 polypeptide” is meant the phosphodiesterases disclosed in Fawcett, 2000 or Yuasa, 2000, as well as any polypeptides encoded by any human allelic variants and any mammalian homologues having PDE11A activity. As used herein, the term “homologue” means a protein that is evolutionarily related to and shares substantial structural and functional similarity with a reference protein in a different species (e.g., human and mouse PDE11A polypeptides).

By “PDE11A polypeptide activity” or “PDE11A polypeptide-like activity” or “PDE11A activity” is meant the hydrolysis of cAMP or cGMP by a polypeptide encoded by a PDE11A gene. Such activity in a cell can be modulated at the level of PDE11A expression (e.g., by changing the amount of polypeptide that is effectively present within a cell) or by modifying the particular functional characteristics of each PDE11A polypeptide molecule (e.g., by changing the Kₘ of hydrolysis for the mutated polypeptide).

By “modulates” is meant increases or decreases (including a complete elimination).

By an agent that is “selective” for modulating PDE11A activity is meant an agent that primarily effects PDE11A while producing little effect on another PDE at the same concentration of agent. For example, if an agent is selective for inhibiting PDE11A, the concentration at which 50% inhibition occurs (IC50) of the agent for PDE11A is at least 20-fold, more preferably, at least 30-fold, even more preferably, at least 50-fold, and, even more preferably, at least 100-fold, lower in concentration as compared to the IC50 for one or more PDEs from the group of PDEs 1-10. Preferably, the agent is selective for PDE11A as compared to PDE7-10, and, even more preferably, as compared to all of PDEs 1-10, especially PDE3-6.

Sexual Dysfunction

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al 1999). FSD is best defined as the difficulty or inability of a woman to find satisfaction in sexual expression. Male sexual dysfunction (MSD) is generally associated with erectile dysfunction, also known as male erectile dysfunction (MED) (Benet et al 1994—Male Erectile dysfunction assessment and treatment options. Comp. Ther. 20: 669-673.).

The agents of the invention are particularly beneficial for the prophylaxis and/or treatment of female sexual dysfunction (FSD), specifically female sexual arousal disorder (FSAD), female orgasmic disorder (FOD) or hypoactive sexual desire disorder (HSDD).

Female Sexual Dysfunction (FSD)

The categories of female sexual dysfunction (FSD) are best defined by contrasting them to the phases of normal female sexual response: desire, arousal and orgasm (see S R Leiblum, (1998), Definition and Classification of Female Sexual Disorders, Int. J. Impotence Res., 10, S104-S106). Desire or libido is the drive for sexual expression. Its manifestations often include sexual thoughts either when in the company of an interested partner or when exposed to other erotic stimuli. Arousal includes the vascular response to sexual stimulation, an important component of which is genital engorgement and increased vaginal lubrication, elongation of the vagina and increased genital sensation/sensitivity and a subjective excitement response. Orgasm is the release of sexual tension that has culminated during arousal. Hence, FSD occurs when a woman has an absent, inadequate or unsatisfactory response in any one or more of these phases, usually desire, arousal or orgasm.

The American Psychiatric Association classifies female sexual dysfunction (FSD) into four classes: FSAD, hypoactive sexual desire disorder (HSDD), female orgasmic disorder (FOD), and sexual pain disorders (e.g. dyspareunia and vaginismus) [see the American Psychiatric Association's Diagnostic and Statistical Manual of Mental Disorders, 4th Edition (DSM-IV)].

DSM-IV defines the four classes as follows:

HSDD—Persistently or recurrently deficient (or absent) sexual fantasies and desire for sexual activity. The judgement of deficiency or absence is made by the clinician, taking into account factors that affect functioning, such as age and the context of the persons life.

FSAD—Persistent or recurrent inability to attain, or to maintain until completion of the sexual activity, an adequate lubrication-swelling response of sexual excitement.

FOD—Persistent or recurrent delay in, or absence of, orgasm following a normal sexual excitement phase. Women exhibit wide variability in the type or intensity of stimulation that triggers orgasm. The diagnosis of FOD should be based on the clinician's judgement that the woman's orgasmic capacity is less than would be reasonable for her age, sexual experience, and the adequacy of the sexual stimulation she receives.

Sexual Pain Disorders such as dyspareunia (recurrent or persistent genital pain associated with sexual intercourse) and vaginismus (recurrent or persistent involuntary spasm of the musculature of the outer third of the vagina that interferes with sexual intercourse).

Hypoactive Sexual Desire Disorder (HSDD)

HSDD is present if a woman has no or little desire to be sexual, and has no or few sexual thoughts or fantasies. This type of FSD can be caused by low testosterone levels, due either to natural menopause or to surgical menopause. Other causes in both pre-menopausal woman (i.e. woman who are pre-menopausal and who have not have hysterectomies) as well as post-menopausal women include illness, medications, fatigue, depression and/or anxiety. Factors having a potential (conscious or sub-conscious) psychological impact such as relationship difficulties or religious factors may be related to the presence of/development of HSDD in females.

Significant HSDD as defined herein means a level of HSDD, which causes some degree of distress to the female subject. Preferably significant HSDD means a level of HSDD which causes some degree of distress and is measurable. More preferably significant HSDD means a level of HSDD which causes some degree of distress and is measurable as a score of less than or equal to 15 on the desire domain (SFQ scale).

Concurrent HSDD as defined herein means a female subject with FSAD who is absent of desire or has significantly low levels of desire, either as a normal (for that subject) or when compared to previous function (for that subject) or not, or has no or few sexual thoughts or fantasies.

It is to be understood that a female subject with FSAD and HSDD whose symptoms of HSDD are on occasion moderated by psychological factors, such that on a rare occasion such a subject may experience a slight increase in her desire for activity but that, in general and overall her function is deficient compared to her previous level (or her normal level) should be classified as suffering from HSDD.

It is to be understood that concurrent significant HSDD as defined herein does not encompass female subjects with situational HSDD. Female subjects with situational HSDD as defined herein includes female subjects who are normally able to become aroused and who normally experience satisfactory levels of sexual desire yet are occasionally unable to experience any satisfactory levels of sexual desire as a direct or indirect response to external factors (such as partner specific HSDD).

Female Sexual Arousal Disorder (FSAD)

The Diagnostic and Statistical Manual (DSM) IV of the American Psychiatric Association defines Female Sexual Arousal Disorder (FSAD) as being:

“ . . . a persistent or recurrent inability to attain or to maintain until completion of the sexual activity adequate lubrication-swelling response of sexual excitement. The disturbance must cause marked distress or interpersonal difficulty . . . ”.

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, per- and post-menopausal (± hormone replacement therapy (HRT)) women. It is associated with concomitant disorders such as depression, cardiovascular diseases, diabetes and urogenital (UG) disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

It has recently been hypothesised that there is a vascular basis for at least a proportion of patients with symptoms of FSAD (Goldstein et al., Int. J. Impot. Res., 10, S84-S90, 1998) with animal data supporting this view (Park et al., Int. J. Impot. Res., 9, 27-37, 1997).

Drug candidates for treating FSAD, which are under investigation for efficacy, are primarily erectile dysfunction therapies that promote circulation to male genitalia. They consist of two types of formulation, oral or sublingual medications (Apomorphine, Phentolamine, phosphodiesterase type 5 (PDE5) inhibitors, e.g. Sildenafil), and prostagiandin (PGE₁) that are injected or administered transurethrally in men and topically to the genitalia in women.

Some agents of the present invention are advantageous by providing a means for restoring a normal sexual arousal response—namely increased genital blood flow leading to vaginal, clitoral and labial engorgement. This will result in increased vaginal lubrication via plasma transudation, increased vaginal compliance and increased genital sensitivity. Hence, the present invention provides a means to restore, or potentiate, the normal sexual arousal response.

By female genitalia herein we mean: “The genital organs consist of an internal and external group. The internal organs are situated within the pelvis and consist of ovaries, the uterine tubes, uterus and the vagina. The external organs are superficial to the urogenital diaphragm and below the pelvic arch. They comprise the mons pubis, the labia majora and minora pudendi, the clitoris, the vestibule, the bulb of the vestibule, and the greater vestibular glands” (Gray's Anatomy, C. D. Clemente, 13^{th} American Edition).

R. J. Levin teaches us that because “. . . male and female genitalia develop embryologically from the common tissue anlagen, [that] male and female genital structures are argued to be homologues of one another. Thus the clitoris is the penile homologue and the labia homologues of the scrotal sac. . . ” (Levin, R. J. (1991), Exp. Clin. Endocrinol., 98, 61-69).

In summary, FSAD is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants e.g. selective serotonin reuptake inhibitors (SSRIs) or antihypertensive agents.

Female (Sexual) Orgasmic Disorder (FSOD or FOD)

FOD is the persistent or recurrent difficulty, delay in or absence of attaining orgasm following sufficient sexual stimulation and arousal, which causes personal distress.

Sexual Pain Disorders

Sexual pain disorders (includes dyspareunia and vaginismus) are characterised by pain resulting from penetration and sexual activity and may be caused by medications which reduce lubrication, endometriosis, pelvic inflammatory disease, inflammatory bowel disease or urinary tract problems.

Other features and advantages of the invention will be apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. Additional guidance with respect to making and using nucleic acids and polypeptides is found in standard textbooks of molecular biology, protein science, and immunology (see, e.g., Davis et al., Basic Methods in Molecular Biology, Elsevir Sciences Publishing, Inc., New York, N.Y., 1986; Hames et al., Nucleic Acid Hybridization, IL Press, 1985; Molecular Cloning, Sambrook et al., Current Protocols in Molecular Biology, Eds. Ausubel et al., John Wiley and Sons; Current Protocols in Human Genetics, Eds. Dracopoli et al., John Wiley and Sons; Current Protocols in Protein Science, Eds. John E. Coligan et al., John Wiley and Sons; and Current Protocols in Immunology, Eds. John E. Coligan et al., John Wiley and Sons). All publications mentioned herein are incorporated by reference in their entireties.

### DESCRIPTION OF THE FIGURES

FIG. 1 shows a schematic depicting an exemplary PDE11A gene targeting or knockout (KO) construct which undergoes homologous recombination with a murine PDE11A sequence and deletes gene sequence corresponding to nucleotides 560-575 of the human cDNA sequence in which the ATG start site begins at position 57, replacing it with the LacZNeo sequence. The deleted portion of the gene comprises sequence encoding the catalytic domain of PDE11A. The sequences of the homology arms that flank the LacZ/Neo sequence are designated SEQ ID NO: 7 and SEQ ID NO: 8. SS refers to intron splice sites within the gene. pA refers to a polyadenylation signal.

FIG. 2A shows an exemplary human PDE11A sequence (SEQ ID NOS: 1 and 2) used to generate PCR primers (SEQ ID NOS: 3 and 4) for use in obtaining the corresponding murine gene sequence by PCR amplification.

FIG. 2B shows the murine PDE11A gene PCR product generated using the primers shown in FIG. 2A. These sequences are examples of murine PDE11A sequences that can be used to design the homology arms of a PDE11A targeting construct, as shown in FIG. 1.

FIG. 3 shows an ethidium bromide-stained agarose gel illustrating the ablation of the PDE11 mRNA in the testis of PDE11 knockout (KO) mice. 5 μg of testis total RNA, extracted from 4 wild type (WT;=WT1, WT2, WT3 and WT4) and 4 PDE11 knockout mice (KO; =KO1, KO2, KO3 and KO4), was subjected to reverse transcription followed by PCR. Using PDE11-specific primers (designed across the site of insertion), the 4 wild type samples show the presence of the intact PDE11 transcript. This transcript is not detected in the PDE11 knockout samples. β-actin controls show equal loading of cDNA.

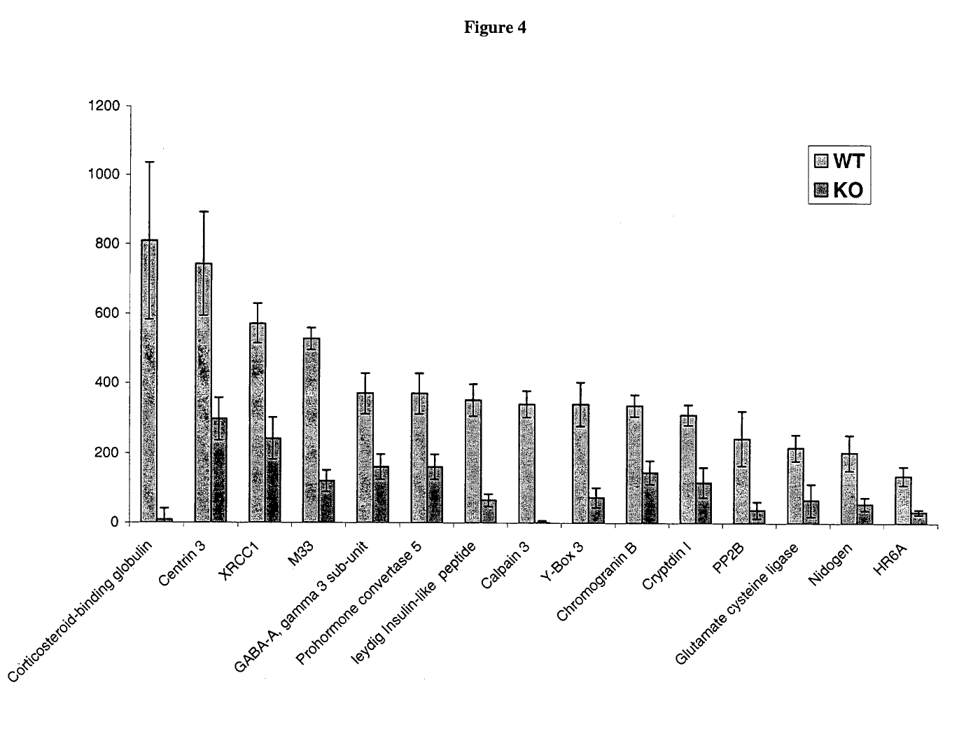

FIG. 4 shows a histogram illustrating the fifteen genes (see later) which show down-regulation of expression in the testis of PDE11 knockout (KO) mice (n=4) compared to wild type (WT) animals (n=4). The histogram bars are average values and error bars are ±1 standard deviation.

FIG. 5 shows a histogram illustrating the six genes (see later) which show up-regulation of expression in the testis of PDE11 knockout (KO) mice (n=4) compared to wild type (WT) animals (n=4). The histogram bars are average values and error bars are ±1 standard deviation.

FIG. 6 shows the degree of capacitation in pre-ejaculated epididymal sperm suspensions obtained from PDE11 knockout (KO) and wild type (WT) mice. The extent of capacitation was measured using chlorotetracycline (CTC) and the degree of capacitation was assessed in control and adenosine-5′-triphosphate (ATP)-treated samples. All data is expressed as mean +standard error of the mean (s.e.m.), n=3, ***=P<0.001, Student's t-test). Control pre-ejaculated spermatozoa obtained from the PDE11 KO animals displayed a significantly higher extent of capacitation compared with sperm obtained from WT mice (White bars, P<0.001, Student's t-test). ATP (2.5 mM) significantly increased the number of capacitated sperm in all murine spermatozoa samples after 1 hr incubation (Grey bars, P<0.001, Student's t-test) whereas only ATP (1.0 mM) significantly increased the number of capacitated sperm in spermatozoa obtained from PDE11 KO mice (Black bars, P<0.001, Student's t-test).

FIGS. 7A and 7B show diagrams that illustrate graphically the relationship between PDE11A modulation and its effects on spermatozoa capacitation (male), sexual desire/libido, sexual arousal and orgasm. FIG. 7A shows that a decrease (↓) in PDE11A activity (e.g. by PDE11A inhibition or ablation) results in a decrease in prolactin (PRL) levels, an increase (↑) in growth hormone (GH) levels, an increase in spermatozoa capacitation, an increase in sexual desire (libido), a decrease in sexual arousal, and a decrease in orgasm. FIG. 7B shows that an increase in PDE11A activity has the opposite effect.

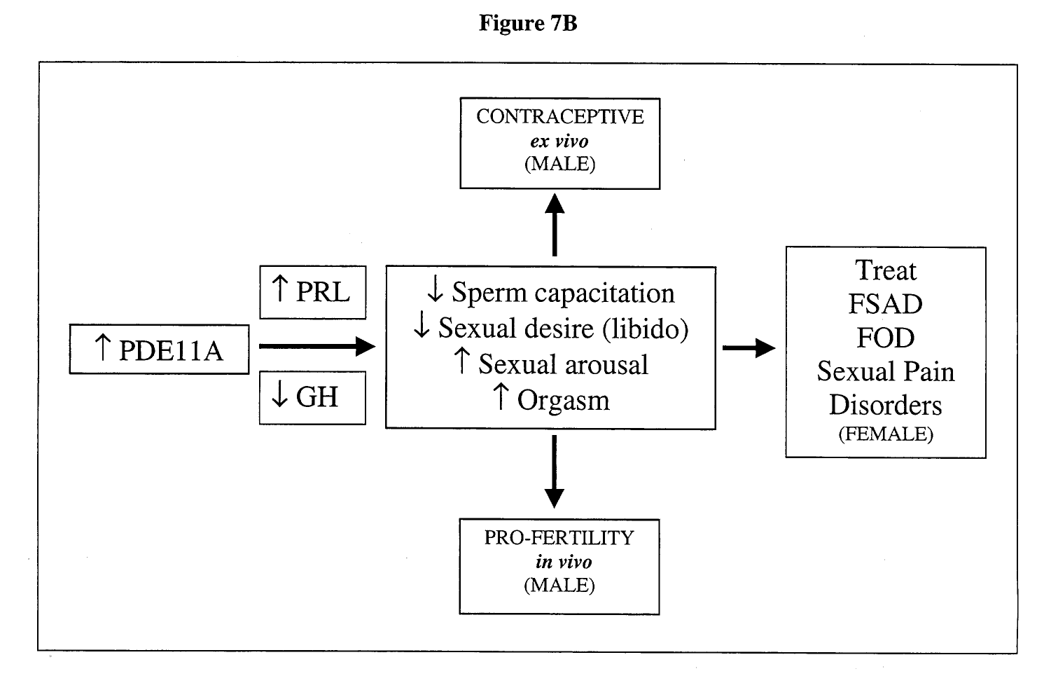

FIG. 8 shows immunostaining of human pituitary for PDE11A using the affinity-purified, polyclonal antiserum EPH-3 (Immunohistochemistry (IHC) Method 1—see Examples). Positive staining appears black against the grey in counter-stain; AH=adenohypophysis (anterior pituitary), NH=neurohypophysis (posterior pituitary).

FIG. 9 shows immunostaining of human pituitary (83-year old male) for PDE11A using the affinity-purified, polyclonal antiserum EPH-3 (Immunohistochemistry (IHC) Method 2—see Examples). Positive staining appears as black deposit; AF=acidophils, B=basophils.

FIG. 10 shows a graph illustrating the trend of age (of mice—both PDE11 knockout (♦) and wild type (□)) and prolactin levels.

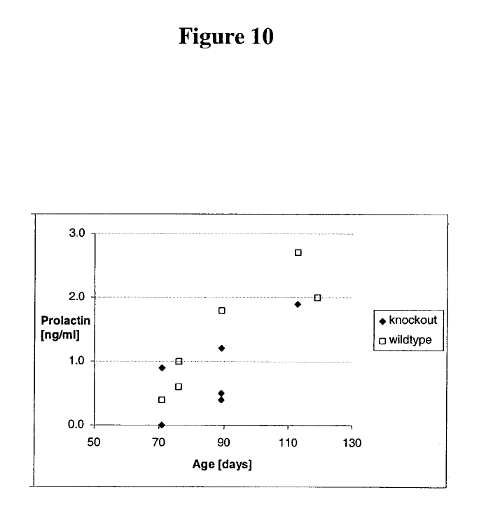

### DETAILED DESCRIPTION

Herein are described genetically-modified animal cells and genetically-modified non-human mammals containing a disrupted PDE11A gene, as well as assays for identifying PDE11A function in the cells and tissues that normally express PDE11A.

Based upon studies of genetically-modified mice homozygous for a PDE11A disruption (PDE11A −/−), we have discovered that PDE11A plays a role in stimulating spermatogenesis, inhibiting spermatozoa capacitation, increasing/normalizing sexual arousal (female) and increasing/normalizing orgasm (female). Accordingly, the present invention provides methods for impacting or affecting, e.g., stimulating or inhibiting, spermatozoa capacitation in a mammal by administering an agent that decreases or increases PDE11A activity, respectively.

1. Genetically-Modified Non-human Mammals and Animal Cells

The genetically-modified, non-human mammals and genetically-modified animal cells, including human cells, described herein are heterozygous or homozygous for a modification that functionally disrupts the PDE11A gene. The animal cells may be derived by genetically engineering cells in culture, or, in the case of non-human mammalian cells, the cells may be isolated from genetically-modified, non-human mammals.

The PDE11A gene locus is functionally disrupted by one of the several techniques for genetic modification known in the art, including chemical mutagenesis (Rinchik, Trends in Genetics 7: 15-21, 1991, Russell, Environmental & Molecular Mutagenesis 23 (Suppl. 24) 23-29, 1994), irradiation (Russell, supra), transgenic expression of PDE11A gene antisense RNA, either alone or in combination with a catalytic RNA ribozyme sequence (Luyckx et al., Proc. Natl. Acad. Sci. 96: 12174-79, 1999; Sokol et al., Transgenic Research 5: 363-71, 1996; Efrat et al., Proc. Natl. Acad. Sci. USA 91: 2051-55, 1994; Larsson et al., Nucleic Acids Research 22: 2242-48, 1994) and, as further discussed below, the disruption of the PDE11A gene by the insertion of a foreign nucleic acid sequence into the PDE11A gene locus. Preferably, the foreign sequence is inserted by homologous recombination or by the insertion of a viral vector. Most preferably, the method of PDE11A gene disruption is homologous recombination and includes a deletion of a portion of the endogenous PDE11A gene sequence.

The integration of the foreign sequence functionally disrupts the PDE11A gene through one or more of the following mechanisms: by interfering with the PDE11A gene transcription or translation process (e.g., by interfering with promoter recognition, or by introducing a transcription termination site or a translational stop codon into the PDE11A gene); or by distorting the PDE11A gene coding sequence such that it no longer encodes a PDE11A polypeptide with normal function (e.g., by inserting a foreign coding sequence into the PDE11A gene coding sequence, by introducing a frameshift mutation or amino acid(s) substitution, or, in the case of a double crossover event, by deleting a portion of the PDE11A gene coding sequence that is required for expression of a functional PDE11A protein).

To insert a foreign sequence into a PDE11A gene locus in the genome of a cell, the foreign DNA sequence is introduced by any suitable method well known in the art such as electroporation, calcium-phosphate precipitation, retroviral infection, microinjection, biolistics, liposome transfection, DEAE-dextran transfection, or transferrinfection (see, e.g., Neumann et al., EMBO J. 1: 841-845, 1982; Potter et al., Proc. Natl. Acad. Sci USA 81: 7161-65, 1984; Chu et al., Nucleic Acids Res. 15: 1311-26, 1987; Thomas and Capecchi, Cell 51: 503-12, 1987; Baum et al., Biotechniques 17: 1058-62, 1994; Biewenga et al., J. Neuroscience Methods 71: 67-75, 1997; Zhang et al., Biotechniques 15: 868-72, 1993; Ray and Gage, Biotechniques 13: 598-603, 1992; Lo, Mol. Cell. Biol. 3: 1803-14, 1983; Nickoloff et al., Mol. Biotech. 10: 93-101, 1998; Linney et al., Dev. Biol. (Orlando) 213: 207-16, 1999; Zimmer and Gruss, Nature 338: 150-153, 1989; and Robertson et al., Nature 323: 445-48, 1986). The preferred method for introducing foreign DNA into a cell is electroporation.

A. Homologous Recombination

The method of homologous recombination targets the PDE11A gene for disruption by introducing a PDE11A gene targeting vector into a cell containing a PDE11A gene. The ability of the vector to target the PDE11A gene for disruption stems from using a nucleotide sequence in the vector that is homologous to the PDE11A gene. This homology region facilitates hybridization between the vector and the endogenous sequence of the PDE11A gene. Upon hybridization, the probability of a crossover event between the targeting vector and genomic sequences greatly increases. This crossover event results in the integration of the vector sequence into the PDE11A gene locus and the functional disruption of the PDE11A gene.

As those skilled in the art will appreciate, general principles regarding the construction of vectors used for targeting are reviewed in Bradley et al. (Biotechnol. 10: 534, 1992). Two different exemplary types of vector can be used to insert DNA by homologous recombination: an insertion vector or a replacement vector. An insertion vector is circular DNA that contains a region of PDE11A gene homology with a double stranded break. Following hybridization between the homology region and the endogenous PDE11A gene, a single crossover event at the double stranded break results in the insertion of the entire vector sequence into the endogenous gene at the site of crossover.

The more preferred vector to use for homologous recombination is a replacement vector, which is collinear rather than circular. Replacement vector integration into the PDE11A gene requires a double crossover event, i.e. crossing over at two sites of hybridization between the targeting vector and the PDE11A gene. This double crossover event results in the integration of vector sequence that is sandwiched between the two sites of crossover into the PDE11A gene and the deletion of the corresponding endogenous PDE11A gene sequence that originally spanned between the two sites of crossover (see, e.g., Thomas and Capecchi et al., Cell 51: 503-12, 1987; Mansour et al., Nature 336: 348-52, 1988; Mansour et al., Proc. Natl. Acad. Sci. USA 87: 7688-7692, 1990; and Mansour, GATA 7: 219-227, 1990).

A region of homology in a targeting vector is generally at least 100 nucleotides in length. Most preferably, the homology region is at least 1-5 kilobases (Kb) in length. There is no demonstrated minimum length or minimum degree of relatedness required for a homology region. However, one skilled in the art will recognize that targeting efficiency for homologous recombination will generally correspond with the length and the degree of relatedness between the targeting vector and the PDE11A gene locus. In the case where a replacement vector is used, and a portion of the endogenous PDE11A gene is deleted upon homologous recombination, an additional consideration is the size of the deleted portion of the endogenous PDE11A gene. If this portion of the endogenous PDE11A gene is greater than 1 Kb in length, then a targeting cassette with regions of homology that are longer than 1 Kb is recommended to enhance the efficiency of recombination. Further guidance regarding the selection and use of sequences effective for homologous recombination is described in the literature (see, e.g., Deng and Capecchi, Mol. Cell. Biol. 12: 3365-3371, 1992; Bollag et al., Annu. Rev. Genet. 23: 199-225, 1989; and Waldman and Liskay, Mol. Cell. Biol. 8: 5350-5357, 1988).

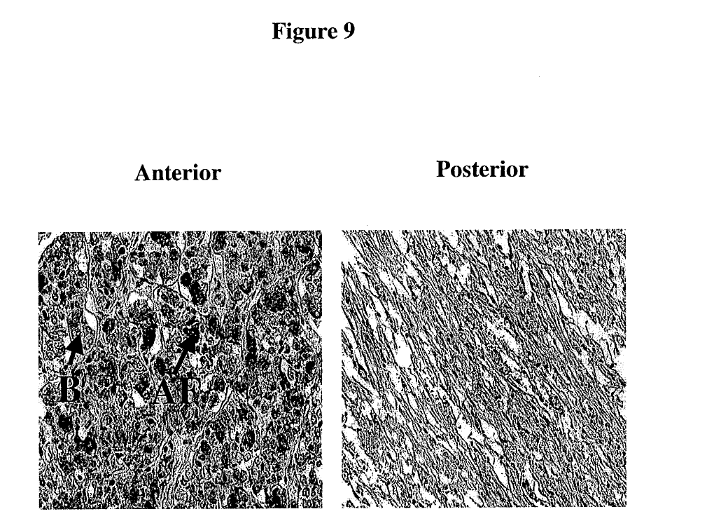

A wide variety of cloning vectors may be used as vector backbones in the construction of the PDE11A gene targeting vectors of the present invention, including pBluescript-related plasmids (e.g., Bluescript KS+11), pQE70, pQE60, pQE-9, pBS, pD10, phagescript, phiX174, pBK Phagemid, pNH8A, pNH16a, pNH18Z, pNH46A, ptrc99a, pKK223-3, pKK233-3, pDR540, and pRIT5 PWLNEO, pSV2CAT, pXT1, pSG (Stratagene), pSVK3, PBPV, PMSG, and pSVL, pBR322 and pBR322-based vectors, pBM9, pBR325, pKH47, pBR328, pHC79, phage Charon 28, pKB11, pKSV-10, pK19 related plasmids, pUC plasmids, and the pGEM series of plasmids. These vectors are available from a variety of commercial sources (e.g., Boehringer Mannheim Biochemicals, Indianapolis, Ind.; Qiagen, Valencia, Calif.; Stratagene, La Jolla, Calif.; Promega, Madison, Wis.; and New England Biolabs, Beverly, Mass.). However, any other vectors, e.g. plasmids, viruses, or parts thereof, may be used for propagation as long as they are replicable and viable in the desired host. The vector may also comprise sequences which enable it to replicate in the host whose genome is to be modified. The use of such a vector can expand the interaction period during which recombination can occur, increasing the efficiency of targeting (see Molecular Biology, ed. Ausubel et al, Unit 9.16, Fig. 9.16.1).

The specific host employed for propagating the targeting vectors of the present invention is not critical. Examples include E. coli K12 RR1 (Bolivar et al., Gene 2: 95, 1977), E. coli K12 HB101 (ATCC No. 33694), E. coli MM21 (ATCC No. 336780), E. coli DH1 (ATCC No. 33849), E. coli strain DH5α, and E. coli STBL2. Alternatively, hosts such as C. cerevisiae or B. subtilis can be used. The above-mentioned hosts are available commercially (e.g., Stratagene, La Jolla, Calif.; and Life Technologies, Rockville, Md.).

To create the targeting vector, a PDE11A gene targeting construct is added to an above-described vector backbone. The PDE11A gene targeting constructs of the invention have at least one PDE11A gene homology region. To make the PDE11A gene homology regions, a PDE11A gene-related sequence is used as a basis for producing polymerase chain reaction (PCR) primers. These primers are used to amplify the desired region of the PDE11A sequence by high fidelity PCR amplification (Mattila et al., Nucleic Acids Res. 19: 4967, 1991; Eckert and Kunkel 1: 17, 1991; and U.S. Pat. No. 4,683,202). The genomic sequence is obtained from a genomic clone library or from a preparation of genomic DNA, preferably from the animal species that is to be targeted for PDE11A gene disruption. PDE11A gene-related sequences have been reported in, e.g., Fawcett, 2000, (human PDE11A1, GenBank Accession No. AJ251509), WO 00/40733 (human PDE11A1 and PDE11A2), and Yuasa, 2000 (human PDE11A3 and PE11A4, GenBank Accession Nos. AB036704 and AB038041).

Preferably, the targeting constructs described herein also include an exogenous nucleotide sequence encoding a positive marker protein. The stable expression of a positive marker after vector integration confers an identifiable characteristic on the cell without compromising cell viability. Therefore, in the case of a replacement vector, the marker gene is positioned between two flanking homology regions so that it integrates into the PDE11A gene following the double crossover event.

It is preferred that the positive marker protein is a selectable protein; the stable expression of such a protein in a cell confers a selectable phenotypic characteristic, i.e., the characteristic enhances the survival of the cell under otherwise lethal conditions. Thus, by imposing the selectable condition, one can isolate cells that stably express the positive selectable marker from other cells that have not successfully integrated the vector sequence on the basis of viability. Examples of positive selectable marker proteins (and their agents of selection) include Neo (G418 or kanamycin), Hyg (hygromycin), HisD (histidinol), Gpt (xanthine), Ble (bleomycin), and Hprt (hypoxanthine) (see, e.g., Capecchi and Thomas, U.S. Pat. No. 5,464,764, and Capecchi, Science 244: 1288-92, 1989). Other positive markers that may also be used as an alternative to a selectable marker include reporter proteins such as β-galactosidase, firefly luciferase, or green fluorescent protein (see, e.g., Current Protocols in Cytometry, Unit 9.5, and Current Protocols in Molecular Biology, Unit 9.6, John Wiley & Sons, New York, N.Y., 2000).

The above-described positive selection scheme does not distinguish between cells that have integrated the vector by targeted homologous recombination at the PDE11A gene locus versus random, non-homologous integration of vector sequence into any chromosomal position. Therefore, when using a replacement vector for homologous recombination, it is also preferred to include a nucleotide sequence encoding a negative selectable marker protein. Expression of a negative selectable marker causes a cell expressing the marker to lose viability when exposed to a certain agent (i.e., the marker protein becomes lethal to the cell under certain selectable conditions). Examples of negative selectable markers (and their agents of lethality) include herpes simplex virus thymidine kinase (gancyclovir or 1,2-deoxy-2-fluoro-α-d-arabinofuransyl-5-iodouracil), Hprt (6-thioguanine or 6-thioxanthine), and diphtheria toxin, ricin toxin, and cytosine deaminase (5-fluorocytosine).

The nucleotide sequence encoding the negative selectable marker is positioned outside of the two homology regions of the replacement vector. Given this positioning, cells will only integrate and stably express the negative selectable marker if integration occurs by random, non-homologous recombination; homologous recombination between the PDE11A gene and the two regions of homology in the targeting construct excludes the sequence encoding the negative selectable marker from integration. Thus, by imposing the negative condition, cells that have integrated the targeting vector by random, non-homologous recombination lose viability.

A combination of positive and negative selectable markers is a preferred selection scheme for making the genetically-modified non-human mammals and animal cells of the invention because a series of positive and negative selection steps can be designed to more efficiently select only those cells that have undergone vector integration by homologous recombination, and, therefore, have a potentially disrupted PDE11A gene. Further examples of positive-negative selection schemes, selectable markers, and targeting constructs are described, for example, in U.S. Pat. No. 5,464,764, WO 94/06908, and Valancius and Smithies, Mol. Cell. Biol. 11: 1402, 1991.

In order for a marker protein to be stably expressed upon vector integration, the targeting vector may be designed so that the marker coding sequence is operably linked to the endogenous PDE11A gene promoter upon vector integration. Expression of the marker is then driven by the PDE11A gene promoter in cells that normally express PDE11A gene. Alternatively, each marker in the targeting construct of the vector may contain its own promoter that drives expression independent of the PDE11A gene promoter. This latter scheme has the advantage of allowing for expression of markers in cells that do not typically express the PDE11A gene (Smith and Berg, Cold Spring Harbor Symp. Quant. Biol. 49: 171, 1984; Sedivy and Sharp, Proc. Natl. Acad. Sci. (USA) 86: 227: 1989; Thomas and Capecchi, Cell 51: 503, 1987).

Exogenous promoters that can be used to drive marker gene expression include cell-specific or stage-specific promoters, constitutive promoters, and inducible or regulatable promoters. Non-limiting examples of these promoters include the herpes simplex thymidine kinase promoter, cytomegalovirus (CMV) promoter/enhancer, SV40 promoters, PGK promoter, PMC1-neo, metallothionein promoter, adenovirus late promoter, vaccinia virus 7.5K promoter, avian beta globin promoter, histone promoters (e.g., mouse histone H3-614), beta actin promoter, neuron-specific enolase, muscle actin promoter, and the cauliflower mosaic virus 35S promoter (see, generally, Sambrook et al., Molecular Cloning, Vols. I-III, Cold Spring Harbor Laboratory Press, Cold Spring Harbor, N.Y., 1989, and Current Protocols in Molecular Biology, John Wiley & Sons, New York, N.Y., 2000; Stratagene, La Jolla, Calif.).

To confirm whether cells have integrated the vector sequence into the targeted PDE11A gene locus, primers or genomic probes that are specific for the desired vector integration event can be used in combination with PCR or Southern blot analysis to identify the presence of the desired vector integration into the PDE11A gene locus (Erlich et al., Science 252: 1643-51, 1991; Zimmer and Gruss, Nature 338: 150, 1989; Mouellic et al., Proc. Natl. Acad. Sci. (USA) 87: 4712, 1990; and Shesely et al., Proc. Natl. Acad. Sci. (USA) 88: 4294, 1991).

B. Gene Trapping

Another method available for inserting a foreign nucleic acid sequence into the PDE11A gene locus to functionally disrupt the PDE11A gene is gene trapping. This method takes advantage of the cellular machinery present in all mammalian cells that splices exons into mRNA to insert a gene trap vector coding sequence into a gene in a random fashion. Once inserted, the gene trap vector creates a mutation that may functionally disrupt the trapped PDE11A gene. In contrast to homologous recombination, this system for mutagenesis creates largely random mutations. Thus, to obtain a genetically-modified cell that contains a functionally disrupted PDE11A gene, cells containing this particular mutation must be identified and selected from a pool of cells that contain random mutations in a variety of genes.

Gene trapping systems and vectors have been described for use in genetically modifying murine cells and other cell types (see, e.g., Allen et al., Nature 333: 852-55, 1988; Bellen et al., Genes Dev. 3: 1288-1300, 1989; Bier et al., Genes Dev. 3: 1273-1287, 1989; Bonnerot et al., J. Virol. 66: 4982-91, 1992; Brenner et al., Proc. Nat. Acad. Sci. USA 86: 5517-21, 1989; Chang et al., Virology 193: 737-47, 1993; Friedrich and Soriano, Methods Enzymol. 225: 681-701, 1993; Friedrich and Soriano, Genes Dev. 5: 1513-23, 1991; Goff, Methods Enzymol. 152: 469-81, 1987; Gossler et al., Science 244: 463-65, 1989; Hope, Develop. 113: 399-408, 1991; Kerr et al., Cold Spring Harb. Symp. Quant. Biol. 2: 767-776, 1989; Reddy et al., J. Virol. 65: 1507-1515, 1991; Reddy et al., Proc. Natl. Acad. Sci. U.S.A. 89: 6721-25, 1992; Skarnes et al., Genes Dev. 6: 903-918, 1992; von Melchner and Ruley, J. Virol. 63: 3227-3233, 1989; and Yoshida et al., Transgen. Res. 4: 277-87, 1995).

Promoter trap (5′ trap) vectors contain, in 5′ to 3′ order, a splice acceptor sequence followed by an exon, which is typically characterized by a translation initiation codon and open reading frame and/or an internal ribosome entry site. In general, these promoter trap vectors do not contain promoters or operably linked splice donor sequences. Consequently, after integration into the cellular genome of the host cell, the promoter trap vector sequence intercepts the normal splicing of the upstream gene and acts as a terminal exon. Expression of the vector coding sequence is dependent upon the vector integrating into an intron of the disrupted gene in the proper reading frame. In such a case, the cellular splicing machinery splices exons from the trapped gene upstream of the vector coding sequence (Zambrowicz et al., WO 99/50426).

An alternative method for producing an effect similar to the above-described promoter trap vector is a vector that incorporates a nested set of stop codons present in, or otherwise engineered into, the region between the splice acceptor of the promoter trap vector and the translation initiation codon or polyadenylation sequence. The coding sequence can also be engineered to contain an independent ribosome entry site (IRES) so that the coding sequence will be expressed in a manner largely independent of the site of integration within the host cell genome. Typically, but not necessarily, an IRES is used in conjunction with a nested set of stop codons.

Another type of gene trapping scheme uses a 3′ gene trap vector. This type of vector contains, in operative combination, a promoter region, which mediates expression of an adjoining coding sequence, the coding sequence, and a splice donor sequence that defines the 3′ end of the coding sequence exon. After integration into a host cell genome, the transcript expressed by the vector promoter is spliced to a splice acceptor sequence from the trapped gene that is located downstream of the integrated gene trap vector sequence. Thus, the integration of the vector results in the expression of a fusion transcript comprising the coding sequence of the 3′ gene trap cassette and any downstream cellular exons, including the terminal exon and its polyadenylation signal. When such vectors integrate into a gene, the cellular splicing machinery splices the vector coding sequence upstream of the 3′ exons of the trapped gene. One advantage of such vectors is that the expression of the 3′ gene trap vectors is driven by a promoter within the gene trap cassette and does not require integration into a gene that is normally expressed in the host cell (Zambrowicz et al., WO 99/50426). Examples of transcriptional promoters and enhancers that may be incorporated into the 3′ gene trap vector include those discussed above with respect to targeting vectors.

The viral vector backbone used as the structural component for the promoter or 3′ gene trap vector may be selected from a wide range of vectors that can be inserted into the genome of a target cell. Suitable backbone vectors include, but are not limited to, herpes simplex virus vectors, adenovirus vectors, adeno-associated virus vectors, retroviral vectors, lentiviral vectors, pseudorabies virus, alpha-herpes virus vectors, and the like. A thorough review of viral vectors, in particular, viral vectors suitable for modifying non-replicating cells and how to use such vectors in conjunction with the expression of an exogenous polynucleotide sequence, can be found in Viral Vectors: Gene Therapy and Neuroscience Applications, Eds. Caplitt and Loewy, Academic Press, San Diego, 1995.

Preferably, retroviral vectors are used for gene trapping. These vectors can be used in conjunction with retroviral packaging cell lines such as those described in U.S. Pat. No. 5,449,614. Where non-murine mammalian cells are used as target cells for genetic modification, amphotropic or pantropic packaging cell lines can be used to package suitable vectors (Ory et al., Proc. Natl. Acad. Sci., USA 93: 11400-11406, 1996). Representative retroviral vectors that can be adapted to create the presently described 3′ gene trap vectors are described, for example, in U.S. Pat. No. 5,521,076.

The gene trapping vectors may contain one or more of the positive marker genes discussed above with respect to targeting vectors used for homologous recombination. Similar to their use in targeting vectors, these positive markers are used in gene trapping vectors to identify and select cells that have integrated the vector into the cell genome. The marker gene may be engineered to contain an IRES so that the marker will be expressed in a manner largely independent of the location in which the vector has integrated into the target cell genome.

Given that gene trap vectors will integrate into the genome of infected host cells in a fairly random manner, a genetically-modified cell having a disrupted PDE11A gene must be identified from a population of cells that have undergone random vector integration. Preferably, the genetic modifications in the population of cells are of sufficient randomness and frequency such that the population represents mutations in essentially every gene found in the cell's genome, making it likely that a cell with a disrupted PDE11A gene will be identified from the population (see Zambrowicz et al., WO 99/50426; Sands et al., WO 98/14614).

Individual mutant cell lines containing a disrupted PDE11A gene are identified in a population of mutated cells using, for example, reverse transcription and PCR to identify a mutation in a PDE11A gene sequence. This process can be streamlined by pooling clones. For example, to find an individual clone containing a disrupted PDE11A gene, RT-PCR is performed using one primer anchored in the gene trap vector and the other primer located in the PDE11A gene sequence. A positive RT-PCR result indicates that the vector sequence is encoded in the PDE11A gene transcript, indicating that PDE11A gene has been disrupted by a gene trap integration event (see, e.g., Sands et al., WO 98/14614).

C. Temporal, Spatial, and Inducible PDE11A Gene Disruptions

Herein, in certain disclosures, a functional disruption of the endogenous PDE11A gene occurs at specific developmental or cell cycle stages (temporal disruption) or in specific cell types (spatial disruption). In other disclosures herein, the PDE11A gene disruption is inducible when certain conditions are present. A recombinase excision system, such as a Cre-Lox system, may be used to activate or inactivate the PDE11A gene at a specific developmental stage, in a particular tissue or cell type, or under particular environmental conditions. Generally, methods utilizing Cre-Lox technology are carried out as described by Torres and Kuhn, Laboratory Protocols for Conditional Gene Targeting, Oxford University Press, 1997. Methodology similar to that described for the Cre-Lox system can also be employed utilizing the FLP-FRT system. The FLP-FRT system and further guidance regarding the use of recombinase excision systems for conditionally disrupting genes by homologous recombination or viral insertion are provided, for example, in U.S. Pat. No. 5,626,159, U.S. Pat. No. 5,527,695, U.S. Pat. No. 5,434,066, WO 98/29533, Orban et al., Proc. Nat. Acad. Sci. USA 89: 6861-65, 1992; O'Gorman et al., Science 251: 1351-55, 1991; Sauer et al., Nucleic Acids Research 17: 147-61, 1989; Barinaga, Science 265: 26-28, 1994; and Akagi et al., Nucleic Acids Res. 25: 1766-73, 1997. More than one recombinase system can be used to genetically modify a non-human mammal or animal cell.

When using homologous recombination to disrupt the PDE11A gene in a temporal, spatial, or inducible fashion, using a recombinase system such as the Cre-Lox system, a portion of the PDE11A gene coding region is replaced by a targeting construct comprising the PDE11A gene coding region flanked by loxP sites. Non-human mammals and animal cells carrying this genetic modification contain a functional, loxP-flanked PDE11A gene. The temporal, spatial, or inducible aspect of the PDE11A gene disruption is caused by the expression pattern of an additional transgene, a Cre recombinase transgene, that is expressed in the non-human mammal or animal cell under the control of the desired spatially-regulated, temporally-regulated, or inducible promoter, respectively. A Cre recombinase targets the loxP sites for recombination. Therefore, when Cre expression is activated, the LoxP sites undergo recombination to excise the sandwiched PDE11A gene coding sequence, resulting in a functional disruption of the PDE11A gene (Rajewski et al., J. Clin. Invest. 98: 600-03,1996; St.-Onge et al., Nucleic Acids Res. 24: 3875-77, 1996; Agah et al., J. Clin. Invest. 100: 169-79, 1997; Brocard et al., Proc. Natl. Acad. Sci. USA 94: 14559-63, 1997; Feil et al., Proc. Natl. Acad. Sci. USA 93: 10887-90, 1996; and Kuhn et al., Science 269: 1427-29, 1995).

A cell containing both a Cre recombinase transgene and loxP-flanked PDE11A gene can be generated through standard transgenic techniques or, in the case of genetically-modified, non-human mammals, by crossing genetically-modified, non-human mammals wherein one parent contains a loxP flanked PDE11A gene and the other contains a Cre recombinase transgene under the control of the desired promoter. Further guidance regarding recombinase systems and specific promoters useful to temporally, spatially, or conditionally disrupt the PDE11A gene is found, for example, in Sauer, Meth. Enz. 225: 890-900, 1993, Gu et al., Science 265: 103-06, 1994, Araki et al., J. Biochem. 122: 977-82, 1997, Dymecki, Proc. Natl. Acad. Sci. 93: 6191-96, 1996, and Meyers et al., Nature Genetics 18: 136-41, 1998.

An inducible disruption of the PDE11A gene can also be achieved by using a tetracycline responsive binary system (Gossen and Bujard, Proc. Natl. Acad. Sci. USA 89: 5547-51, 1992). This system involves genetically modifying a cell to introduce a Tet promoter into the endogenous PDE11A gene regulatory element and a transgene expressing a tetracycline-controllable repressor (TetR). In such a cell, the administration of tetracycline activates the TetR which, in turn, inhibits PDE11A gene expression and, therefore, functionally disrupts the PDE11A gene (St.-Onge et al., Nucleic Acids Res. 24: 3875-77, 1996, U.S. Pat. No. 5,922,927).

The above-described systems for temporal, spatial, and inducible disruptions of the PDE11A gene can also be adopted when using gene trapping as the method of genetic modification, for example, as described in WO 98/29533, for example.

D. Creating Genetically-Modified, Non-human Mammals and Animal Cells

The above-described methods for genetic modification can be used to functionally disrupt a PDE11A gene in virtually any type of somatic or stem cell derived from an animal. Genetically-modified animal cells described herein include, but are not limited to, mammalian cells, including human cells, and avian cells. These cells may be derived from genetically engineering any animal cell line, such as culture-adapted, tumorigenic, or transformed cell lines, or they may be isolated from a genetically-modified, non-human mammal carrying the desired PDE11A genetic modification.

The cells may be heterozygous or homozygous for the disrupted PDE11A gene. To obtain cells that are homozygous for the PDE11A gene disruption (PDE11A−/−), direct, sequential targeting of both alleles can be performed. This process can be facilitated by recycling a positive selectable marker. According to this scheme the nucleotide sequence encoding the positive selectable marker is removed following the disruption of one allele using the Cre-Lox P system. Thus, the same vector can be used in a subsequent round of targeting to disrupt the second PDE11A gene allele (Abuin and Bradley, Mol. Cell. Biol. 16: 1851-56, 1996; Sedivy et al., T. I. G. 15: 88-90, 1999; Cruz et al., Proc. Natl. Acad. Sci. (USA) 88: 7170-74, 1991; Mortensen et al., Proc. Natl. Acad. Sci. (USA) 88: 7036-40, 1991; te Riele et al., Nature (London) 348: 649-651, 1990).

An alternative strategy for obtaining ES cells that are PDE−/− is the homogenotization of cells from a population of cells that is heterozygous for the PDE11A gene disruption (PDE11A±). The method uses a scheme in which PDE11A± targeted clones that express a selectable drug resistance marker are selected against a very high drug concentration; this selection favors cells that express two copies of the sequence encoding the drug resistance marker and are, therefore, homozygous for the PDE11A gene disruption (Mortensen et al., Mol. Cell. Biol. 12: 2391-95, 1992). In addition, genetically-modified animal cells can be obtained from genetically-modified PDE11A−/− non-human mammals that are created by mating non-human mammals that are PDE11A± in germline cells, as further discussed below.

Following the genetic modification of the desired cell or cell line, the PDE11A gene locus can be confirmed as the site of modification by PCR analysis according to standard PCR or Southern blotting methods known in the art (see, e.g., U.S. Pat. No. 4,683,202; and Erlich et al., Science 252: 1643, 1991). Further verification of the functional disruption of the PDE11A gene may also be made if PDE11A gene messenger RNA (mRNA) levels and/or PDE11A polypeptide levels are reduced in cells that normally express the PDE11A gene. Measures of PDE11A gene mRNA levels may be obtained by using reverse transcriptase mediated polymerase chain reaction (RT-PCR), Northern blot analysis, or in situ hybridization. The quantification of PDE11A polypeptide levels produced by the cells can be made, for example, by standard immunoassay methods known in the art. Such immunoassays include, but are not limited to, competitive and non-competitive assay systems using techniques such as radioimmunoassays, ELISA (enzyme-linked immunosorbent assay), “sandwich” immunoassays, immunoradiometric assays, gel diffusion precipitin reactions, immunodiffusion assays, in situ immunoassays (using colloidal gold, enzymatic, or radioisotope labels, for example), Western blots, 2-dimensional gel analysis, precipitation reactions, immunofluorescence assays, protein A assays, and immunoelectrophoresis assays.

Preferred genetically-modified animal cells are ES cells and ES-like cells. These cells are derived from the pre-implantation embryos and blastocysts of various species, such as mice (Evans et al., Nature 129:154-156, 1981; Martin, Proc. Natl. Acad. Sci., USA, 78: 7634-7638, 1981), pigs and sheep (Notanianni et al., J. Reprod. Fert. Suppl., 43: 255-260, 1991; Campbell et al., Nature 380: 64-68, 1996) and primates, including humans (Thomson et al., U.S. Pat. No. 5,843,780; Thomson et al., Science 282: 1145-1147, 1995; and Thomson et al., Proc. Natl. Acad. Sci. USA 92: 7844-7848, 1995).

These types of cells are pluripotent. That is, under proper conditions, they differentiate into a wide variety of cell types derived from all three embryonic germ layers: ectoderm, mesoderm and endoderm. Depending upon the culture conditions, a sample of ES cells can be cultured indefinitely as stem cells, allowed to differentiate into a wide variety of different cell types within a single sample, or directed to differentiate into a specific cell type, such as macrophage-like cells, neuronal cells, cardiomyocytes, adipocytes, smooth muscle cells, endothelial cells, skeletal muscle cells, keratinocytes, and hematopoietic cells, such as eosinophils, mast cells, erythroid progenitor cells, or megakaryocytes. Directed differentiation is accomplished by including specific growth factors or matrix components in the culture conditions, as further described, for example, in Keller et al., Curr. Opin. Cell Biol. 7: 862-69, 1995, Li et al., Curr. Biol. 8: 971, 1998, Klug et al., J. Clin. Invest. 98: 216-24, 1996, Lieschke et al., Exp. Hematol. 23: 328-34, 1995, Yamane et al., Blood 90: 3516-23, 1997, and Hirashima et al., Blood 93: 1253-63, 1999.

The particular embryonic stem cell line that is used for genetic modification is not critical; exemplary murine ES cell lines include AB-1 (McMahon and Bradley, Cell 62:1073-85, 1990), E14 (Hooper et al., Nature 326: 292-95, 1987), D3 (Doetschman et al., J. Embryol. Exp. Morph. 87: 27-45, 1985), CCE (Robertson et al, Nature 323: 445-48, 1986), RW4 (Genome Systems, St. Louis, Mo.), and DBA/1lacJ (Roach et al., Exp. Cell Res. 221: 520-25, 1995). Genetically-modified murine ES cells may be used to generate genetically-modified mice, according to published procedures (Robertson, 1987, Teratocarcinomas and Embryonic Stem Cells: A Practical Approach, Ed. E. J. Robertson, Oxford: IRL Press, pp. 71-112, 1987; Zjilstra et al., Nature 342: 435-438, 1989; and Schwartzberg et al., Science 246: 799-803, 1989).

Following confirmation that the ES cells contain the desired functional disruption of the PDE11A gene, these ES cells are then injected into suitable blastocyst hosts for generation of chimeric mice according to methods known in the art (Capecchi, Trends Genet. 5: 70, 1989). The particular mouse blastocysts employed herein are not critical. Examples of such blastocysts include those derived from C57BL/6 mice, C57BL/6 Albino mice, Swiss outbred mice, CFLP mice, and MFI mice. Alternatively, ES cells may be sandwiched between tetraploid embryos in aggregation wells (Nagy et al., Proc. Natl. Acad. Sci. USA 90: 8424-8428, 1993).

The blastocysts or embryos containing the genetically-modified ES cells are then implanted in pseudopregnant female mice and allowed to develop in utero (Hogan et al., Manipulating the Mouse Embryo: A Laboratory Manual, Cold Spring Harbor Laboratory, 1988; and Teratocarcinomas and Embryonic Stem Cells: A Practical Approach, E. J. Robertson, ed., IRL Press, Washington, D.C., 1987). The offspring born to the foster mothers may be screened to identify those that are chimeric for the PDE11A gene disruption. Generally, such offspring contain some cells that are derived from the genetically-modified donor ES cell as well as other cells from the original blastocyst. In such circumstances, offspring may be screened initially for mosaic coat color, where a coat color selection strategy has been employed, to distinguish cells derived from the donor ES cell from the other cells of the blastocyst. Alternatively, DNA from tail tissue of the offspring can be used to identify mice containing the genetically-modified cells.

The mating of chimeric mice that contain the PDE11A gene disruption in germ line cells produces progeny that possess the PDE11A gene disruption in all germ line cells and somatic cells. Mice that are heterozygous for the PDE11A gene disruption can then be crossed to produce homozygotes (see, e.g., U.S. Pat. No. 5,557,032, and U.S. Pat. No. 5,532,158).

An alternative to the above-described ES cell technology for transferring a genetic modification from a cell to a whole animal is to use nuclear transfer. This method can be employed to make other genetically-modified, non-human mammals besides mice, for example, sheep (McCreath et al., Nature 29: 1066-69, 2000; Campbell et al., Nature 389: 64-66, 1996; and Schnieke et al., Science 278: 2130-33, 1997) and calves (Cibelli et al., Science 280: 1256-58, 1998). Briefly, somatic cells (e.g., fibroblasts) or pluripotent stem cells (e.g., ES-like cells) are selected as nuclear donors and are genetically-modified to contain a functional disruption of the PDE11A gene. When inserting a DNA vector into a somatic cell to mutate the PDE11A gene, it is preferred that a promoterless marker be used in the vector such that vector integration into the PDE11A gene results in expression of the marker under the control of the PDE11A gene promoter (Sedivy and Dutriaux, T. I. G. 15: 88-90, 1999; McCreath et al., Nature 29: 1066-69, 2000). Nuclei from donor cells which have the appropriate PDE11A gene disruption are then transferred to fertilized or parthenogenetic oocytes that are enucleated (Campbell et al., Nature 380: 64, 1996; Wilmut et al., Nature 385: 810, 1997). Embryos are reconstructed, cultured to develop into the morula/blastocyst stage, and transferred into foster mothers for full term in utero development.

The ambit of the description herein also encompasses the progeny of the genetically-modified, non-human mammals and-genetically-modified animal cells. While the progeny are heterozygous or homozygous for the genetic modification that functionally disrupts the PDE11A gene, they may not be genetically identical to the parent non-human mammals and animal cells due to mutations or environmental influences that may occur in succeeding generations at other loci in addition to the genetic modification presently described.

E. “Humanized” Non-human Mammals and Animal Cells

The genetically-modified non-human mammals and non-human animal cells described herein, and which contain a disrupted endogenous PDE11A gene, can be further modified to express the human PDE11A sequence (referred to herein as “humanized”). The human PDE11A sequence is disclosed, for example, in Fawcett, 2000, Phillips et al., WO 00/40733, and GenBank Accession No. AJ251509.

A preferred method for humanizing cells involves substituting the human PDE11A sequence for the endogenous PDE11A by homologous recombination. The targeting vectors are similar to those traditionally used as knock-out vectors with respect to the 5′ and 3′ homology arms and positive/negative selection schemes. However, the vectors also include sequences that, upon recombination, insert the human PDE11A coding sequence in exchange for the endogenous sequence, or affect base pair changes, codon substitutions, or exon substitutions, that modify the endogenous sequence such that the sequence encodes the human PDE11A instead of the endogenous wild type sequence. Once homologous recombinants have been identified, it is possible to excise any inserted selection-based sequences (e.g., neo) by using Cre or Flp-mediated site-directed recombination (Dymecki, Proc. Natl. Acad. Sci. 93: 6191-96, 1996).

It is preferred that the human PDE sequence be positioned directly downstream of the endogenous translation start site. This positioning preserves the endogenous temporal and spatial expression patterns of the PDE11A gene. The human sequence can comprise the whole genomic PDE11A sequence, or the full length human cDNA sequence with a polyA tail attached at the 3′ end for proper processing (Shiao et al., Transgenic Res. 8: 295-302, 1999). Further guidance regarding these methods of genetically modifying cells and non-human mammals to replace expression of an endogenous gene with its human counterpart is found, for example, in Sullivan et al., J. Biol. Chem. 272: 17972-80, 1997, Reaume et al., J. Biol. Chem. 271: 23380-88, 1996, and Scott et al., U.S. Pat. No. 5,777,194.

Another method for creating such “humanized” organisms is a multi-step process involving, first, the creation of PDE11A−/− embryos, and, second, the introduction of a transgene encoding the human sequence into the PDE11A−/− embryos.

F. Example—Generation of PDE11A± and PDE11A−/− Mice

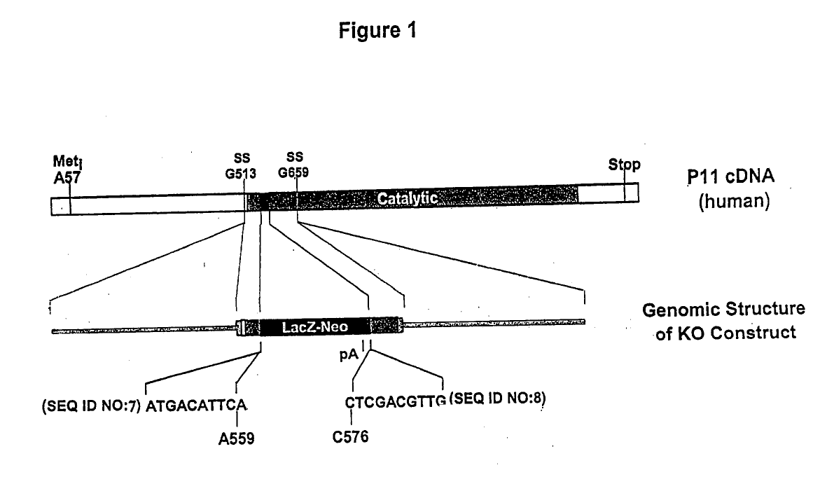

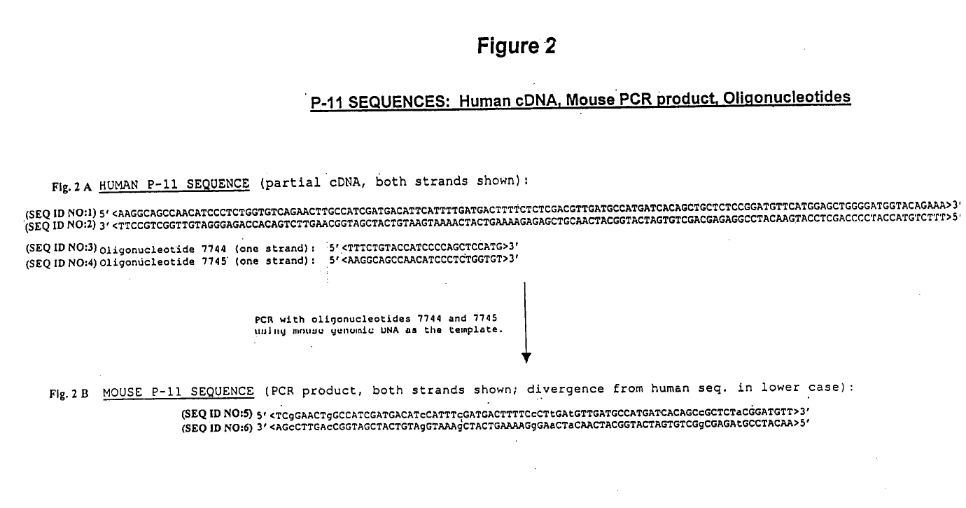

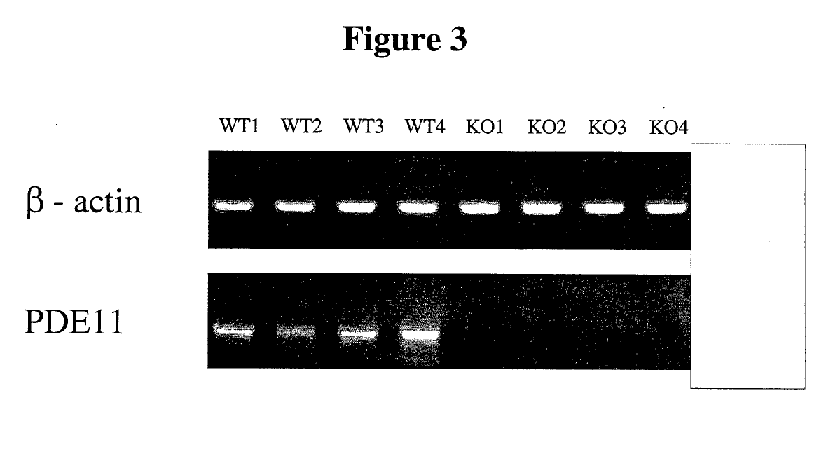

Genetically-modified mouse ES cells (PDE11A±) were generated using the scheme shown in FIG. 1 (DeltaGen, Menlo Park, Calif., USA). A partial cDNA sequence of the human PDE11A gene (FIG. 2A; SEQ ID NOS: 1 and 2) was used to design oligonucleotide strands 7744 and 7745 as shown below.

| | |
|---|---|
| Oligonucleotide 7744- | |
| 5′ TTTCTGTACCATCCCCAGCTCCATG 3′ | (SEQ ID NO: 3) |
| Oligonucleotide 7745- | |
| 5′ AAGGCAGCCAACATCCCTCTGGTGT 3′ | (SEQ ID NO:4) |

PCR-based amplification of mouse genomic DNA using the above primers generated the product representing the mouse PDE11A sequence shown in FIG. 2B (SEQ ID NOS: 5 and 6). Based upon this genomic sequence, a targeting construct was created which contained two homology arms (each of 10 nucleotides in length) of SEQ ID NOS: 7 and 8, and a LacZ-Neo sequence inserted between the arms, as shown in FIG. 1. DNA containing the targeting construct was inserted into the ES cells by electroporation. Integration of this construct into the murine PDE11A gene in ES R1 cells (Deng et al., Dev. Biol. 185: 42-54, 1997; Udy et al., Exp. Cell Res. 231: 296-301, 1997) resulted in the replacement of the nucleotides 27-42 of SEQ ID NOS: 5 and 6 with the LacZ-Neo gene. ES cells that were neomycin resistant were analyzed by Southern blot to confirm disruption of a PDE11A gene. These targeted ES cells were then used for generation of chimeric mice by injecting the cells into blastocysts and implanting the blastocysts into pseudopregnant female mice (Capecchi et al., Trends Genet. 5: 70, 1989, Hogan et al., Manipulating the Mouse Embryo: A Laboratory Manual, Cold Spring Harbor Laboratory, 1988; and Teratocarcinomas and Embryonic Stem Cells: A Practical Approach, E. J. Robertson, ed., IRL Press, Washington, D.C., 1987). Chimeric mice were then bred with C57BL/6 (Jackson Laboratories, Bar Harbor, Me., USA) mice to create F1 PDE± heterozygotes, which were in turn bred to produce F2 PDE−/− homozygotic mice. The functional disruption of the PDE11A gene in the heterozygotes and homozygotes was confirmed by PCR and Southern blot analysis.

2. Characterization of PDE11A Function and Therapeutic Relevance

Perturbations that modulate PDE11A activity or expression can be induced in cells, tissues, or mammals to determine whether PDE11A modulation produces a physiologically relevant effect or phenotype. One means of inducing such a perturbation is genetically modifying a non-human mammal or animal cell to disrupt the PDE11A gene. Alternatively, an agent that modulates PDE11A activity or expression can be administered to determine its effect on a cell or tissue preparation, or on a mammal.

A. Tissue Expression

Guidance for determining which cells, tissues, or phenotypes to study with respect to PDE11A function is found in the PDE11A expression pattern in species such as mice and humans. In addition to what has previously been described for human PDE11A expression in Fawcett, 2000, (i.e., testis, skeletal muscle, kidney, liver, various glandular tissue (e.g., pituitary, salivary, adrenal, mammary, and thyroid), pancreas, spinal cord, and trachea), we hereby disclose additional tissues that express PDE11A, and further characterize expression in the previously disclosed tissues.

PDE11A expression patterns were determined in formalin-fixed, paraffin-embedded normal human tissues sectioned at a width of 5 μm (Peterborough Hospital Cellular Pathology Services, Peterborough, UK), and paraformaldehyde-fixed, paraffin-embedded normal mouse testis samples sectioned at 7 μM (Cat. No. 69584-3, Novagen, Madison, Wis., USA). The immobilized sections were dewaxed by washing in xylene (5 min.), absolute alcohol (5 min.), and then industrial methylated spirits (5 min.) Afterwards the slides were washed for 5 min. in running water, then treated for antigen retrieval according to one of the following methods: 1) treated with trypsin for 15 min. at 37° C. (Cat. No. T7168, Sigma Chemicals, St. Louis, Mo., USA, 1 tablet/ml water), followed by another wash in running water; or 2) treated with target retrieval solution (Cat. No. S1700, DAKO Corp., Carpinteria, Calif., USA) using the steamer method as per manufacturer's instructions, followed by several washes in tris-buffered saline (TBS) (Cat. No. T6664, Sigma Chemicals, St.

Louis, Mo., USA, 1 sachet/L water). Samples were soaked in fresh TBS and treated with the components of one of two alternative immunohistochemical kits, as indicated below.

Samples were incubated with a primary rabbit anti-PDE11A antibody (1:500 or 1:000 dilution, in TBS/5% non-fat milk for 60 min.) The primary antibody was raised against the human PDE11A (GenBank Accession No. AJ251509) catalytic domain peptide sequence of SAIFDRNRKDELPRL (SEQ ID NO: 9) (Cambridge Research Biochemicals, Cleveland, UK). The antibody detection system included either: 1) an anti-rabbit secondary antibody conjugate to horseradish peroxidase (HRP) and a 3,3′-diaminobenzidine (DAB) chromogen solution (Cat. No. K4010, DAKO EnVision™+System, DAKO Corp.); or 2) an anti-rabbit secondary antibody conjugated to alkaline phosphatase (AP) (Cat. No.-AK5001, ABC-AP kit, Vector Laboratories, Burlingame, Calif., USA) and an AP substrate kit (Cat. No. SK-5100, Vector® Red, Vector Laboratories). The immunohistochemical procedures were conducted according to manufacturers' recommendations. Following incubation with the chromogen, tissues were counterstained with hemotoxylin (Cat. No. H3401, Vector Laboratories). The samples were then rinsed in water and dipped 10 times (10×) in acid rinse (2% v/v glacial acetic acid), followed by 10× in water, 20× in Scott's tap water (Cat. No. 6769002, Shandon Scientific, Runcorn, UK), rinsed 3 min. in water, dehydrated, mounted in DPX (Cat. No. 360294H, BDH Laboratory Supplies, Poole, UK), and sealed with a coverslip.

Strong PDE11A antibody staining was demonstrated in the following cell types and structures: cardiac myocytes in all chambers of the heart; endothelial cells lining the endocardium; medial smooth muscle cells of the cardiac (epicardial and intromyocardial) and systemic vasculature in venules, veins, arterioles, and arteries (particularly in vessels of smaller diameter), vascular endothelial cells; Schwann cells and perineural cells (nerve and vascular staining often correlated in particular tissues, e.g., bladder, testis, and heart); in the testis, in nuclei of spermatogonia, as well as spermocytes and spermatids, interstitial Leydig cells and cells underlying the basement membrane of the seminiferous tubules (fibroblasts), and in the vascular smooth muscle; amine precursor uptake and decarboxylation (APUD) enteroendocrine cells in the colon and rectal glands; neurons throughout the brain (moderate to strong staining), such as in the substantia nigra, amygdyla, nucleus basalis, the area postrema, and in acidophiles (somatotrophs and lactotrophs) of the anterior pituitary (especially in the periphery of the lateral zones); alveolar macrophages in the lung; islet cells in the pancreas; rare cells in the red pulp of the spleen; in numerous adipocytes; in lymphoid tissue; and in samples from cancerous prostate, hyperplastic prostate, and skin with malignant melanoma.

Additional cell types and tissues that showed moderate to weak staining for PDE11A included the following: the bladder urothelium, especially the basal to transitional epithelial layers (in the cytoplasm and, especially, in the nuclei of cells); cells in the exocrine parenchyma; keratinocytes in the basal and prickle layers of the epidermis; the cortex, outer root sheath, and collagenous root sheath of the hair follicle; eccrine glands; occasional Purkinje cells in the cerebellum; ependymal cells, cells in the basal ganglia and striatum; ganglion cells in Auerbach's plexus and Meissner's plexus; synovial cells; hepatocytes; smooth muscle; pleural mesothelium; squamous cells in the vaginal mucosa; lobular and ductal epithelial cells in the breast; vascular and sinusoidal lining cells in the splenic red pulp; sebaceous glands; convoluted tubules and collecting ducts in the kidney; histiocytes, including myointimal histiocytes in atherosclerosis; and mast cells, plasma cells, lymphocytes, and granulocytes.

Further clarification of expression in skeletal muscle showed variable staining, with strong staining of the motor end plates in a few samples. Staining varied from weak to strong in the anterior tibialis myocytes, the extensor digitorum longus, the gastrocnemius, the soleus, and in the vastus lateralis.

Murine tissue expression patterns of PDE11A demonstrated weak expression in testis in the germinal epithelium of the seminiferous tubules (spermatogonia, spermatocytes, spermatids, Leydig cells, and vascular smooth muscle). This expression is particularly prevalent in the spermatocytes.

B. Identification of PDE11A Agonists and Antagonists

One type of screen to identify PDE11A agonists and antagonists is conducted in vitro using native enzymes isolated from tissue, or using recombinant enzymes expressed in transfected host cells, for example, Sf9 insect cells (Fawcett, 2000), yeast cells (Loughney et al., U.S. Pat. No. 5,932,465), or COS-7 cells (Yuasa, 2000). Preferably, the PDE11A enzyme is human. Agents identified as PDE11A agonists or antagonists would likely produce similar effects on any other member of the PDE11 family.

PDE11A activity is measured, for example, as the rate of hydrolysis of an appropriate substrate, [³H]cAMP or [³H]cGMP. Agents that increase or decrease substrate hydrolysis are identified as PDE11A selective agonists (i.e., enhancers) or antagonists (i.e., inhibitors), respectively. This activity is measured, for example, by scintillation proximity assay (SPA)-based methods (Fawcett, 2000; Phillips et al., WO 00/40733, and Thompson et al., Biochem. 18: 5228, 1979 (as modified using product code TRKQ7090/7100, Amersham International Ltd., Buckinghamshire, UK)). Briefly, samples containing the PDE11A enzyme are contacted with a cAMP or cGMP substrate (Sigma Chemical), a portion (e.g., ¼ to ½) of which is ³H-labelled (Amersham). Reactions are conducted in, for example, microtiter plates (e.g., Microfluor® plates, Dynex Technologies, Chantilly, Va., USA), and are terminated by the addition of yttrium silicate SPA beads (Amersham) containing excess unlabeled cyclic nucleotide. After the beads are allowed to settle in the dark, plates are read by a microtiter plate reader (e.g., TopCount®, Packard, Meriden, Conn., USA).

PDE activity may also be assayed by detection of ³²P-phosphate released from ³²P-cAMP or ³²P-cGMP (as described, for example, in Loughney et al., J. Biol. Chem. 271: 796-806, 1996, and Loughney, U.S. Pat. No. 5,932,465), or using antibodies to distinguish between the PDE substrates, cGMP or cAMP, and their hydrolyzed products (using, for example FlashPlate™ technology, NEN® Life Sciences Products, Boston, Mass., USA).

As an alternative to assaying PDE11A catalytic activity, agents can be identified as PDE11A agonists or antagonists if they indirectly modulate PDE11A catalytic activity, for example, via post-translational modification (e.g., phosphorylation), modulation of allosteric ligand binding (e.g., via the GAF domain (Fawcett, 2000)), or by binding to PDE11A themselves at either a catalytic or allosteric regulatory site. Agents that increase PDE11A catalytic activity via these mechanisms are identified as potential agonists; conversely, agents that decrease PDE11A catalytic activity via these mechanisms are potential antagonists. Methods for determining PDE11A phosphorylation and allosteric ligand binding are described in the literature (see, e.g., McAllister-Lucas et al., J. Biol. Chem. 270: 30671-79, 1995, and Corbin et al., Eur. J. Biochem. 267: 2760-67, 2000).

Examples of agents that are screened include, but are not limited to, nucleic acids (e.g., DNA and RNA), carbohydrates, lipids, proteins, peptides, peptidomimetics, small molecules and other compounds. Agents can be selected individually for testing or as part of a library. These libraries are obtained using any of the numerous approaches in combinatorial library methods known in the art, and include: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (e.g., Lam, 1997, Anticancer Drug Des. 12:145; U.S. Pat. No. 5,738,996; and U.S. Pat. No. 5,807,683).

Examples of methods for the synthesis of molecular libraries can be found in the art, for example, in DeWitt et al., 1993, Proc. Natl. Acad. Sci. USA 90:6909, Erb et al., 1994, Proc. Natl. Acad. Sci. USA 91:11422, Zuckermann et al., 1994, J. Med. Chem. 37:2678, Cho et al., 1993, Science 261:1303, Carrell et al., 1994, Angew. Chem. Int. Ed. Engl. 33:2059, Carell et al., 1994, Angew. Chem. Int. Ed. Engl. 33:2061, and Gallop et al., 1994, J. Med. Chem. 37:1233.

Individual agents or libraries of agents may be presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devin, 1990, Science 249:404-406; Cwirla et al., 1990, Proc. Natl. Acad. Sci. USA 87:6378-6382; and Felici, 1991, J. Mol. Biol. 222:301-310).

The effects of the test agents on PDE11A enzymatic kinetics (e.g., Vmax and Km) are determined in vitro, for example, by measuring hydrolysis with a fixed concentration of PDE11A enzyme, a range of substrate concentrations (e.g., 0.10-10 μM), and a time course ranging from, for example, 5-60 minutes. Agents that increase Vmax or decrease Km are identified as agonists. Agents that inhibit PDE11A activity decrease Vₘₐₓ or increase Km and, preferably, have IC50 values in the nanomolar range. Such IC50 determinations involve assaying a fixed amount of purified recombinant or native PDE11A in the presence of various inhibitor concentrations and a low substrate concentration (e.g., 0.1-1.0 μM). Values for samples exposed to inhibitors are converted to percent activity of an uninhibited control (100%) and plotted against inhibitor concentration to find the concentration at which 50% inhibition occurs (IC50).

Preferably, the screen identifies PDE11A agonists and antagonists that are selective for a PDE11A enzyme. For example, a preferred agonist or antagonist is selective for PDE11A by at least 20-fold, more preferably, at least 30-fold, even more preferably, at least 50-fold, and, most preferably, at least 100-fold, as compared one or more PDEs from the group of PDEs 1-10. Preferred agonists and antagonists are selective for PDE11A as compared to one or more of PDE3, PDE4, PDE5, and/or PDE6.

For example, fold selectivity of an antagonist with respect to PDE11A as compared to another PDE is determined by dividing the IC50 value for the other PDE by the IC50 value for PDE11A. Fold selectivity comparisons may involve comparing PDE11A values to previously reported values for other PDEs, or testing the other PDE enzymes concurrently.

As an alternative to in vitro screens, compounds can be screened for PDE11A modulating effects in a cell-based, tissue-based, or whole animal-based assay, using samples that express PDE11A endogenously or as a result of genetic engineering.

In addition to comparing such samples in the presence and absence of the agent, an additional negative control for such assays is testing the agent on an appropriately matched, genetically-modified, PDE11A−/− cell or tissue preparation or non-human mammal. Such samples can be used to verify whether or not any observed effect in PDE11A+/+ samples is mediated by PDE11A.

### EXAMPLE

Identification of PDE11A Antagonists

Agents known to inhibit PDE5 were tested to determine whether they also modulated PDE11A activity according to the method described, e.g., in Fawcett, 2000 and in Phillips et al., WO 00/40733. The IC50 values of the agents for inhibiting PDE11A were compared to the IC50 values for inhibiting PDEs 5-10. PDE5 was isolated from human corpus cavernosum; PDE6 was isolated from bovine retina; and recombinant human PDEs 7-11 were produced by baculovirus expression in Sf9 cells (Fawcett, 2000, and Ballard et al., J. Urol. 159: 2164-71, 1998).

The agents tested included the following: Sildenafil (5-[2-ethoxy-5-(4-methyl-1-piperazinylsulphonyl)phenyl]-1-methyl-3-n-propyl-1,6-dihydro-7H-pyrazolo[4,3-d]pyrimidin-7-one, which is also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756)); Cialis (IC351; (6R,12aR)-6-(1,3-benzodioxol-5-yl)-2,3,6,7,12,12a-hexahydro-2-methyl-pyrazino[1′,2′: 1,6]pyrido[3,4-b]indole-1,4-dione; CAS number: 171488-01-0; see also WO 95/19978), E4021 (1-[4-[(1,3-benzodioxol-5-ylmethyl)amino]-6-chloro-2-quinazolinyl]-4-piperidinecarboxylic acid, monohydrochloride; CAS number: 150452-21-4; see, also, WO 93/07124); and UK-235,187 (6-Chloro-2-(1H-imidazol-1-yl)-N-(phenylmethyl)-,4-quinazolinamine, dihydrochloride; CAS number: 157863-31-5; see, also, EP 579496).

Compounds were dissolved in DMSO (4 mM) and then diluted to 40 μM in buffer B (20 mM Tris-HCl, pH 7.4, 5 mM MgCl₂-hexahydrate, 2 mg/ml bovine serum albumin (BSA)) at 25° C. (all components are available from Sigma Chemicals, St. Louis, Mo.). This 40 μM solution was then serially diluted to prepare a half log dilution over at least 5 points. Samples of each range point (25 μl) were plated in duplicate in microtiter plates (Microfluor® plates, Dynex Technologies, Chantilly, Va.). Samples containing the PDE enzyme were then added to each well. Purified recombinant or native enzyme was diluted 1:500 in buffer B, and 25 μl of this dilution was added to each well. (This amount of enzyme was sufficient to ensure a linear reaction over time and concentration.) The enzyme and compound samples were then pre-incubated for 15 minutes at room temperature.

Substrate was added to each plate in 50 μl samples. Substrate consisted of 90 nM unlabelled cGMP (Sigma Chemicals), 48 nM ³H-GMP (Cat. No. RPNQ0150, Amersham International Ltd.) in buffer A (20 mM Tris-HCl, pH 7.4, 5 mM MgCl₂ hexahydrate) for a final cGMP concentration of 69 nM for PDE11A tests (the substrate concentration for the other PDEs tested was adjusted to be less that or equal to ⅓ Km). Following a 50 minute incubation at room temperature on a plate shaker set to ensure adequate mixing, the reaction was terminated by the addition of yttrium silicate SPA beads (Amersham) containing excess (3 mM) unlabeled cGMP. The plate was then shaken for 15 minutes to ensure even mixing and then beads were allowed to settle for 30 minutes. Plates were read by a microtiter plate reader (TopCount® plate reader, TopCount protocol 50, Packard, Meriden, Conn.).

The agents' IC50 values for inhibiting PDE11A were compared to those for inhibiting PDEs 5-10. As shown in Table 1 below, PDE5 inhibitors Cialis (IC351), E4021, and UK-235,187 demonstrated selectivity for inhibiting PDE11A when compared to PDEs 7-10 (Cialis (IC351) was also selective for PDE11A over PDE6). By contrast, the PDE5 inhibitor Sildenafil was not selective for PDE11A when compared to any of PDEs 5-10.

TABLE 1
IC50

| Compound | PDE5 | PDE6 | PDE7 | | PDE8 | | PDE9 | | PDE10 | | PDE11A |
|---|---|---|---|---|---|---|---|---|---|---|---|
| Sildenafil | 3.5 nM | 37.5 nM | 19.7 | μM | 27.6 | μM | 2.48 | μM | 9.13 | μM | 2.99 μM |
| IC351 | 6.74 nM | 1.5 μM | >100 | μM | >100 | μM | >100 | μM | >100 | μM | 37.0 nM |
| E4021 | 33.4 nM | 4.7 nM | >100 | μM | >100 | μM | >100 | μM | >100 | μM | 572 nM |
| UK-235,187 | 4.64 nM | 1.0 μM | >100 | μM | >100 | μM | >100 | μM | >100 | μM | 46.6 nM |

All results are the geometric mean of at least 3 determinations.

C. Phenotypic Characterization of PDE11A−/− and PDE11A± Mice

The genetically-modified PDE11A−/− and PDE11A± non-human mammals and animal cells described herein that have reduced PDE11A polypeptide activity can be used to identify novel PDE11A-mediated biological functions based upon the appearance of any abnormal phenotypes, for example, as related to decreased spermatogenesis, increased spermatozoa capacitation, NO-mediated bladder relaxation, and vasodilation. Any abnormal phenotypes associated with decreased PDE11A activity establish a basis for identifying and developing PDE11A-targeted therapeutics for preventing or treating PDE11A-related diseases or conditions.

### EXAMPLE

PDE11A−/− mice Demonstrate Reduced Spermatogenesis

Mice homozygous for the PDE gene disruption were compared to wild type mice to identify any abnormal phenotypes at the age of 6-8 weeks. Data was collected from physical examination, necropsy, histology, clinical chemistry, blood chemistry, body weight, organ weight, and the cytological evaluation of bone marrow. For these comparisons, six PDE−/− mice (3 males and 3 females), and four PDE11A+/+ wild-type mice (2 males and 2 females) were studied.

Histological studies revealed changes in the testes of male PDE11A−/− mice. These changes included reduced spermatogenesis, an increased sloughing of spermatogenic epithelial cells, a thinning of the spermatogenic epithelium, a decrease in average seminiferous tubule diameter, a degeneration of the epithelial cells lining the tubule, and increased sloughing of residual bodies. These PDE11A KO mice also demonstrated reduced epididymal sperm, increased multinucleate sperm precursors, and an increased appearance of cell fragments and proteinaceous debris. Other tissues examined were histologically normal. These other tissues included seminal vesicles, salivary gland, thymus gland, pituitary gland, thyroid gland, aorta, heart, liver, gallbladder, esophagus, caecum, colon, rectum, bones, joint tissue, spinal cord, bone marrow, trachea, larynx, skeletal muscle, sciatic nerve, tongue, mammary gland, ovary, uterus, cervix, ocular tissue, and Harderian glands. No consistent or significant differences between the PDE11A−/− mice and control mice were revealed in terms of blood chemistry analysis, organ weight, or body weight.

The abnormal phenotype observed in PDE11A−/− male mice indicates that PDE11A is normally involved in spermatogenesis. Therefore, inhibiting or enhancing PDE11A activity in a male mammal decreases or increases spermatogenesis, respectively. The role of PDE11A in spermatogenesis is further confirmed by PDE11A expression in the germinal epithelium of the seminiferous tubules, particularly in spermatocytes, of wild type mice.

### EXAMPLE

PDE11A−/− mice Demonstrate Increased Capacitation in Spermatozoa

Capacitation (CP) is the process spermatozoa normally undergo during their journey through the female tract to reach and bind to the zona pellucida, prior to fertilising the egg (oocyte), and is an important determinant of fertility. The biochemical events of capacitation that are triggered in post-ejaculation spermatozoa lead to spermatozoa becoming ‘switched on’. This enables sperm to undergo the acrosome reaction and ultimately fertilize an oocyte. Failure for spermatozoa to become capacitated may be an underlying cause of infertility, as could premature termination of capacitation via spontaneous acrosome loss. Pre-ejaculated spermatozoa are uncapacitated and become capacitated after ejaculation. Capacitation of sperm and the triggering of all the downstream consequences of this process are facilitated by the addition of a milieu of bioactive molecules that make up seminal fluid and are mixed with sperm at ejaculation. Whilst many of the constituents of this milieu remain to be elucidated, ATP is known to be an important element. It is well established that sperm physiology, in particular capacitation, is regulated by elevation of intra-spermatozoal cAMP. The phosphodiesterase sub-types that hydrolyze spermatozoal cAMP are uncharacterized. It has been suggested that premature capacitation of spermatozoa can negatively influence their fertilization ability (Thundathil, J., Gil, J., Januskauskas, A., et al. (1999). Relationship between the proportion of capacitated spermatozoa present in frozen-thawed bull semen and fertility with artificial insemination. Int. J. Andrology, 22, 366-373). Capacitated spermatozoa exhibit elevated metabolic rates, increased membrane fluidity and permeability, and if they do not reach the oocyte, they undergo spontaneous acrosome loss. Hence the life-span of capacitated spermatozoa is limited (Thundathil et al, 1999). Thus, prematurely capacitated sperm are less likely to survive until they reach the oocyte and site of fertilization, hence giving rise to reduced fertility or even infertility.

Methodology

Introduction

Tissue sections were examined by immunohistochemistry (IHC) using a specific anti-PDE11 antibody. KO mice were generated using standard techniques, and PDE11 ablation confirmed by Southern blotting as well as reverse transcriptase polymerase chain reaction (RT-PCR) and IHC analysis of the testis. Fresh epididymal sperm were collected from adult mice and tested for capacitation (CP) using a chlorotetracycline assay before and after treatment with ATP—an inducer of CP.

Immunohistochemistry

Formalin-fixed, paraffin-embedded tissue sections were examined by immunohistochemistry (IHC; see later sections entitled Immunohistochemistry (IHC)—Materials and Methods and Immunohistochemistry (IHC) Method 1 for methodology) using a specific anti-peptide antibody (EPH3) directed against the catalytic domain of human and murine PDE11.

Gene Knockout (KO)

PDE11 KO mice were generated using standard techniques and back-crossed with C57BL/6 mice prior to characterisation; PDE11 ablation was confirmed by Southern blotting as well as RT-PCR and IHC analysis of tissues of interest, including testis.

Capacitation Analysis

For capacitation analyses, fresh epididymal spermatozoa were collected from adult (16 week-old) mice into Tyrodes media, and tested in triplicate using a chlorotetracycline (CTC) assay (Fraser, L. R. & Herod, J. E. Expression of capacitation-dependent changes in chlortetracycline fluorescence patterns in mouse spermatozoa requires a suitable glycolysable substrate. J. Reprod. Fert., 1990; 88(2): 611-21) both ± pre-treatment with ATP—an inducer of capacitation—to determine the functional state of the spermatozoa.

Sperm suspensions were prepared by releasing the contents of mouse epididymides from 3 PDE11 knockout (KO) and 3 wild type (WT) mice into Tyrodes media. The extent of capacitation was measured using chlorotetracycline (CTC), 750 μM for 15 min at 37° C. The patterns of CTC fluorescence were observed under microscopy (Reference: Fraser L R et al., 1990, J. Reprod. Fert.—see above). Patterns reflected the functional state of spermatozoa, i.e. capacitated, uncapacitated and acrosome loss.

Results

In normal mouse (adult, n=5) and human testis (17-70 yrs, n=5), PDE11 protein was localised to the spermatogonia, spermatocytes and spermatids (within the seminiferous tubules), as well as the Leydig and vascular smooth muscle cells (i.e. positive staining). The sertoli cells and connective tissue elements stained negative. PDE11 KO mice were viable ( and ), and testes were devoid of PDE11 mRNA and protein expression (n=4; 16 week-old).

The functional status of the spermatozoa is summarized below:

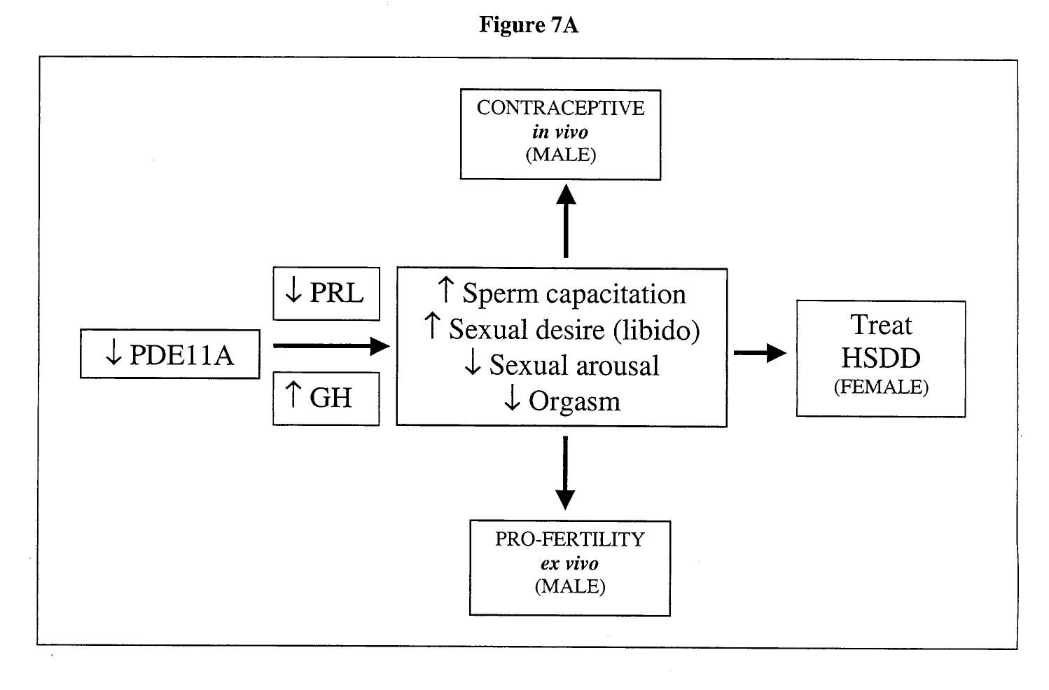

TABLE 2
(n = 3) - see also Figure 6

| | % capacitated sperm (n = 3 animals × | | |
|---|---|---|---|
| Pre- | n = 2/3; mean ± SEM) | | |
| treatment | Wild-type (+/+) | PDE11 KO (−/−) | P (wt vs. KO) |
| None (basal) | 18.8 ± 2.4 | 31.5 ± 3.2 | 0.006 |
| +1mM ATP | 41.4 ± 8.2 | 45.9 ± 7.0 | NS |
| +2.5 mM ATP | 73.5 ± 5.1 | 73.9 ± 5.6 | NS |

TABLE 3
(n = 4)

| | % capacitated sperm (n = 4, ≧2 replicates | | |
|---|---|---|---|
| Pre- | each; mean ± SEM) | | |
| treatment | Wild-type (+/+) | PDE11 KO (−/−) | P (wt vs. KO) |
| None (basal) | 19.4 ± 1.4 | 29.2 ± 2.0 | <0.001 |
| +1 mM ATP | 47.1 ± 5.1 | 48.4 ± 4.0 | NS |
| +2.5 mM ATP | 78.4 ± 3.3 | 77.6 ± 3.8 | NS |

Conclusions

Baseline capacitation, of pre-ejaculated spermatozoa, was found to be significantly higher in PDE11 knockout mice compared with wild type mice (see FIG. 6). This data suggests that there is a significant proportion of premature capacitation in the sperm obtained from PDE11 knockout mice. This premature capacitation may impair the fertilization potential of these spermatozoa. The maximal extent of capacitation was similar between PDE11 knockout mice compared with wild type mice. In both samples, ATP significantly increased the number of capacitated murine spermatozoa after 1 hr incubation.

The data suggest a role for PDE11 in maintaining spermatozoa in an uncapacitated state prior to activation (e.g., ejaculation)—this being consistent with the localisation of PDE11 protein in mouse and man—presumably via an enhanced cyclic nucleotide monophosphate (cNMP) pathway (augmentation of a cAMP and/or cGMP signaling pathway). However, PDE11 does not appear to have a role in the potential for CP upon activation with ATP. Since the proportion of uncapacitated spermatozoa at insemination is known to correlate positively with egg-penetrating capability (Thundathil J et al., 1999, Int. J. Androl.), enhancement of PDE11 activity/signaling may improve in vivo male fertility.

D. Genotypic Characterization of PDE11A−/− and PDE11A± Mice

The genetically-modified PDE11A−/− and PDE11A± non-human mammals and animal cells described herein that have reduced PDE11A polypeptide activity can be used to identify novel PDE11A-mediated biological functions based upon the up- and/or down-regulation of certain genes, for example, as related to spermatogenesis, spermatozoa capacitation, NO-mediated bladder relaxation, and vasodilation. Any abnormal gene expression associated with decreased PDE11A activity establishes a basis for identifying and developing PDE11A-targeted therapeutics for preventing or treating PDE11A-related diseases or conditions.

### EXAMPLE

PDE11A−/− Mice Demonstrate Abnormal Gene Expression Compared to Wild Type Mice

RNA Extraction

Tissue frozen in liquid nitrogen (from the testis of PDE11A−/− knock-out (KO) mice and wild type (WT) mice) was pulverized using a dismembrator (B. Braun Biotech International) and lysed in Buffer RLT (Qiagen, Germany). The solution was homogenized using a Qiashredder (Qiagen) and the RNA extracted using RNeasy columns (Qiagen), following the manufacturer's protocol.

Affymetrix Gene Expression Analysis

Double-stranded cDNA was generated from 10 μg total RNA using Superscript Choice kit (Invitrogen (Life Technologies) Limited, Scotland, UK) with a T7-polyT primer. Approximately 1 μg cDNA was used to generate biotinylated cRNA by in vitro transcription using Bioarray High Yield RNA Transcript Labeling Kit (Enzo, USA). 10 μg fragmented cRNA was hybridized in 100 mM MES, 1M Na⁺, 20 mM EDTA, 0.01% Tween 20, 0.1 mg/ml herring sperm DNA, 0.5 mg/ml acetylated BSA plus 50 pM control oligonucleotide and eukaryotic hybridization controls to Murine Genome U74A arrays (MGU74A arrays—Catalogue Nos.: 900321 or 900322; Affymetrix, Calif., USA) at 45° C. for 16 hours. Arrays were washed using Affymetrix protocols in non-stringent buffer (6×SSPE, 0.01% Tween 20, 0.005% antifoam) at 25° C. and stringent wash buffer (100 mM MES, 0.1 M Na⁺, 0.01% Tween) at 50° C. and stained with streptavidin phycoerythrin (10 μg/ml) including an antibody amplification step. Arrays were scanned using a laser confocal scanner to generate fluorescence intensities. The data were processed using Microarray Analysis Suite version 4.0 (Affymetrix). Approximately 2,600 probe sets identified by Affymetrix as non-functional were masked out of the analysis. The data were scaled to a target intensity of 300.

Data Analysis of the n=4 Data Set

The data was subsequently analyzed using Spotfire Array Explorer 3.0 software (see, for example, U.S. Pat. No. 6,014,661). Genes were initially selected based on expression profiles which were “high in WT/low in KO” (i.e. down-regulated by PDE11 ablation) or “low in WT/high in KO” (i.e. up-regulated by PDE11 ablation). The 1000 genes so selected were then triaged on the basis of expression level and fold change (at least 2-fold between WT and KO)—this removed the majority of the genes. Expressed sequence tag (EST) clusters (function unknown at present) were then also removed. This left 108 genes, either down- or up-regulated which have a known function. Each of these genes were then researched in Medline for an involvement in, inter alia, testis function, cAMP and/or cGMP-mediated signal transduction. Of the 72 down-regulated genes in the KO mice, 15 have some known link to testis. Of the 35 up-regulated genes, 6 have some known link to testis.

Summary of Data Analysis of Genes Down-Regulated in PDE11 KO Testis

500 genes were identified which show similarity to an expression profile of high in WT/low in KO.

Of these, 199 genes were not expressed at significant levels in either WT or KO for the results to be meaningful and were disregarded.

Of the 301 genes left, 170 genes showed less than a 2-fold differential expression between WT and KO. Although statistically all of these genes were significantly differentially expressed to at least a significance of P=0.05, for the purposes of the present analysis they were excluded.

Of the remaining 131 genes, 59 genes were derived from mouse EST clusters and have no identifiable function at present.

Of the remaining 72 genes, 43 genes had no discernible role in, inter alia, testis function based on searches of the literature.

Of the remaining 29 genes, 12 genes were excluded for other reasons.

2 further genes were known to be involved in xenobiotic metabolism.

This left 15 genes (see below), which have varying links to testicular function and are significantly down-regulated in the PDE11 KO testis.

Summary of Data Analysis of Genes Up-Regulated in PDE11 KO Testis

500

genes were identified which show similarity to an expression profile of low in WT/high in KO.

Of these, 269 genes were not expressed at significant levels in either WT or KO for the results to be meaningful and were disregarded.

Of the 231 genes left, 161 genes showed less than a 2-fold differential expression between WT and KO. Although statistically all of these genes were significantly differentially expressed to at least a significance of P=0.05, for the purposes of the present analysis they were excluded.

Of the remaining 70 genes, 35 genes were derived from mouse EST clusters and have no identifiable function at present.

Of the remaining 35 genes, 28 genes had no discernible role in, inter alia, testis function based on searches of the literature.

1 gene was known to be involved in xenobiotic metabolism.

This left 6 genes (see below), which have varying links to testicular function and are significantly up-regulated in the PDE11 KO testis.

Gene Expression

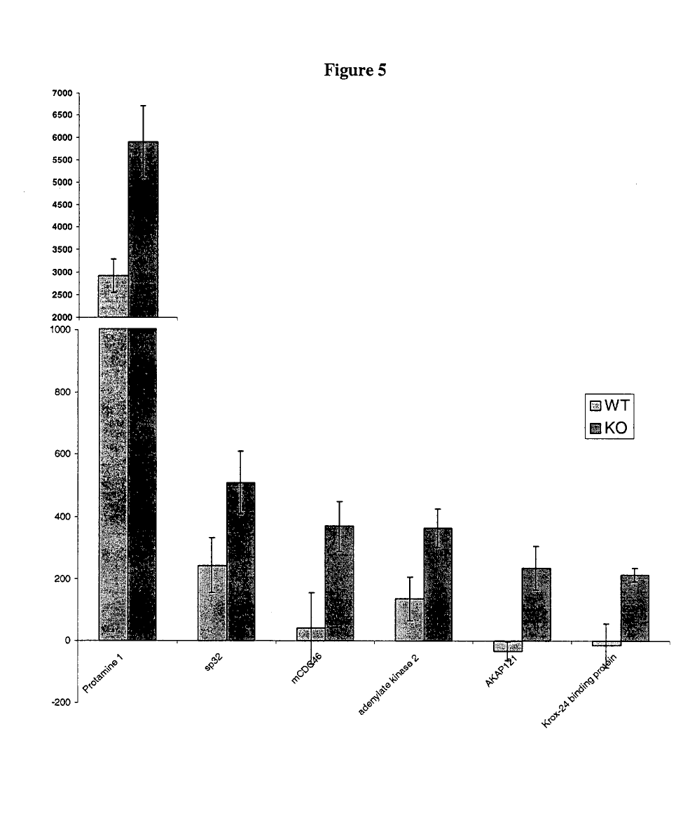

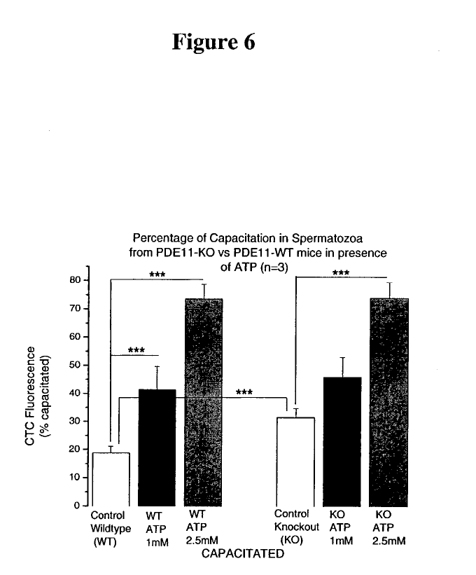

The microarray data showed fifteen genes that were down-regulated in the testis of PDE11 knock-out mice (n=4) compared to wild type animals (n=4) (see FIG. 4) and six genes that were up-regulated in the testis of PDE11 knock-out mice (n=4) compared to wild type animals (n=4) (see FIG. 5).

15 Down-Requlated Genes:

1. Corticosteroid binding globulin is known to be involved in the transport of steroids (e.g. testosterone) and it is related to Serpins. Primary expression is in the liver, but also expressed in testis (Proc. Natl. Acad. Sci. USA 84:5153). Down-regulation of this gene will affect transport of the testosterone secreted by Leydig cells. It could have down-stream affects on spermatid viability or secondary sexual characteristics.

2. Centrin 3 is an essential protein involved in centromere formation and cell cycle progression. Down-regulation of Centrin might affect mitotic turnover and therefore, reduce the numbers of spermatids produced.

3. XRCC1 is involved in DNA strand-break repair, homologous recombination, and sister chromatid exchange. Expression is known not to change over time in mice. Therefore, down-regulation of XRCC1 may cause incorrect repair of DNA during mitosis (Brain Res. 869:105).

4. Chromobox M33 is a transcription factor and is related to Polycomb. Down-regulation of this protein could mimic the sex reversal phenotype seen in the KO mouse.

5. GABA-A, gamma 3 sub-unit is an important structural component of the GABA-A receptor. GABA is known to be required for the acrosome reaction. Down-regulation of this sub-unit may cause inefficient signalling via the GABA-A receptor which may impair the acrosome reaction.

6. Prohormone convertase 5 is thought to activate MIS. Down-regulation of this protease could cause developmental abnormalities associated with improper mullerian duct regression.

7. Leydig Insulin-like peptide is a Relaxin-like factor involved in gubernaculum development. Down-regulation of this gene could mimic aspects of the Ley-L KO mouse e.g. cryptochordism of the testis.

8. Calpain 3 is involved in the acrosome reaction. Other calpains are not down-regulated. Down-regulation of Calpain 3 might affect the acrosome reaction.

9. Y-Box 3 is involved in transcription. Other Y-Box proteins are not down-regulated. As it is suspected to be an import transcription factor in testis, down-regulation could have multiple affects on testicular physiology.

10. Chromogranin B is Involved in secretion from endocrine tissues. Down-regulation of this protein might disrupt secretory pathways of the Leydig cells and thereby causing alterations in the interstitial microenvironment.

11. Cryptdin I is important for defense against infective bacteria. Cryptdins have been shown to be involved in bacterial defense in the testis. Therefore, reduced cryptidin expression could make testis susceptible to infection.

12. PP2B is a phosphatase known to be involved in the Calmodulin-mediated calcium signalling pathway which is key for spermatogenesis. Down-regulation of this protein might cause disturbance to calcium signalling and affect efficient spermatogenesis.

13. Glutamate cysteine ligase is involved in the Gamma glutamyl cycle. As this cycle is known to be essential for spermatogenesis, down-regulation could inhibit this process.

14. Nidogen is involved in adhesion of the peritubular cells to extracellular matrix. Down-regulation of this gene could cause instability in the peritubular cells and improper spermiation and migration.

15. HR6A is involved in Ubiquitin conjugation. Down-regulation of this protein could mimic the compromised spermatogenesis consistent with the phenotype seen in the KO mouse.

6 Up-Regulated Genes:

1. Protamine 1 is a histone like protein which is specific to testis. Changes in protamine 1 regulation is known to cause incorrect condensation of the spermatid nucleus.

2. sp32 is a proacrosin-binding factor. It accelerates activation of proacrosin which could be deleterious to fertilization efficiency.

3. mCDC46 is a DNA replication-licensing factor which is essential for DNA replication. Up-regulation could reflect deleterious changes in the testis.

4. Adenylate kinase 2 is important for nucleotide regulation. Up-regulation could be a response to elevated cAMP levels.

5. AKAP121 is a kinase anchor protein. It is known to be regulated by cAMP and hormones. Up-regulation could reflect deleterious changes in testicular microenvironment.

6. Krox-24 binding protein binds with Zinc-finger transcription factor which activates EGR. Increased expression may contribute to the infertility of the knock-out mice.

E. Identification of PDE11A Function Using PDE11A Agonists and Antagonists

Compounds identified as modulators of PDE11A, more preferably, compounds that are identified as selective PDE11A modulators, can be administered to cells or tissue preparations that express PDE11A, or to whole animals, to identify PDE11A function based upon changes that occur in response to compound administration.

For example, given PDE11A expression in bladder urothelium, it is a candidate as a regulator of nitric oxide (NO)-mediated inhibition of bladder and bladder nerve fiber excitability. This NO-mediated effect is described, e.g., in Ozawa et al., J. Urol. 162: 2211, 1999, Pandita et al., J. Urol. 545-550, 2000, and Burnett et al., Nat. Med. 3: 571-74, 1997. To characterize the role of PDE11A in bladder contractility, the effects of a PDE11A antagonist can be studied in a model of oxyhemoglobin-induced (Pandita, supra) or cyclophosphamide-induced (Ozawa, supra), bladder hyperactivity. Experimental animals (e.g., rats) are administered oxyhemoglobin (intravesically, Sigma Chemical) or cyclophosphamide (intraperitoneally) in combination with a PDE11A antagonist dissolved, e.g., in DMSO and diluted by an appropriate buffer. The PDE11A antagonist is administered by an appropriate route (e.g., intravesically, intraperitoneally, or intravenously).

PDE11A is identified as playing a role in regulating NO-mediated inhibition of bladder and bladder nerve fiber excitability in test animals if a PDE11A antagonist causes a decrease in neuronal firing in afferent neurons innervating the bladder, a decrease in bladder contraction frequency, a decrease in micturition pressure, and/or a decrease basal pressure, as compared to control animals not administered the antagonist. The measures of bladder function are made according to standard methods described in the literature (e.g. Pandita (supra), and Ozawa (supra)). As an additional control to confirm that the observed effect was mediated by inhibiting PDE11A, appropriately matched, genetically-modified non-human mammals containing a disrupted PDE11A gene (preferably, PDE11A−/−) may be tested.

When testing the effect of an agent that modulates PDE11A activity or expression, it is preferred to use tissue or cell samples that express human PDE11A, such as those derived from human cell lines or from a primary human tissue preparation. Alternatively, such tissue or cell samples may be obtained from a PDE11A humanized non-human mammal or animal cell. Similarly, one preferred test animal for PDE11A functional studies is a genetically-modified PDE11A humanized mammal.

F. PDE11A Expression and its Role in Modulating Secretion of Prolactin and/or Growth Hormone

lmmunohistochemistry (IHC)

Materials and Methods

An anti-human PDE11A polyclonal antiserum (EPH-3) was raised in rabbits against the synthetic peptide SAIFDRNRKDELPRL, which corresponds to amino acid residues 410-424 of human PDE11A1, and subsequently affinity-purified as previously described (Fawcett et al., 2000).

lmmunohistochemistry (IHC) Method 1:

5 μm human tissue sections, embedded in paraffin and immobilised on APES (3-aminopropyltriethoxysilane)-coated slides, were purchased from Peterborough Hospital Cellular Pathology Services (Peterborough, UK). All tissues had been obtained by surgery, or within 24 h of post-mortem, and fixed in formalin. These samples were de-waxed and taken to water, then antigens were retrieved at 37° C. for 15 min with 1 mg/ml trypsin in a buffer of 200 mM Tris (pH7.7) and 4 mM CaCl₂. Following a brief wash in water, sections were then processed for immunodetection using the DAKO Rabbit EnVision™+system (Cat# K4010) with 3,3′-diaminobenzidine (DAB) as the substrate chromogen. For specific protein detection, sections were incubated with EPH-3 antibody diluted 1:1000 in tris-buffered saline (TBS) containing 5% (w/v) non-fat milk powder, and incubated for 1 hour at room temperature. Negative controls were included: both pre-immune serum and antibody mixed with blocking peptide gave the same result. Sections were counter-stained in haematoxylin and permanently mounted in DPX.

Immunohistochemistry (IHC) Method 2:

Antibody EPH-3 (diluted 1:500 or 1:1000) was used as the primary antibody, and the principal detection system consisted of a Vector ABC-AP Kit (AK5001, anti-rabbit secondary antibody) with a Vector Red substrate kit, which was used to produce a fuschia-colored red deposit (SK-5100). Tissues were also stained with a positive control antibody (CD31) to ensure that the tissue antigens were preserved and accessible for immunocytochemical analysis. Only tissues that stained positive for CD31 were chosen for the remainder of this study. Negative controls consisted of performing the entire immunocytochemistry procedure on adjacent sections in the absence of primary antibody.

Expression Data

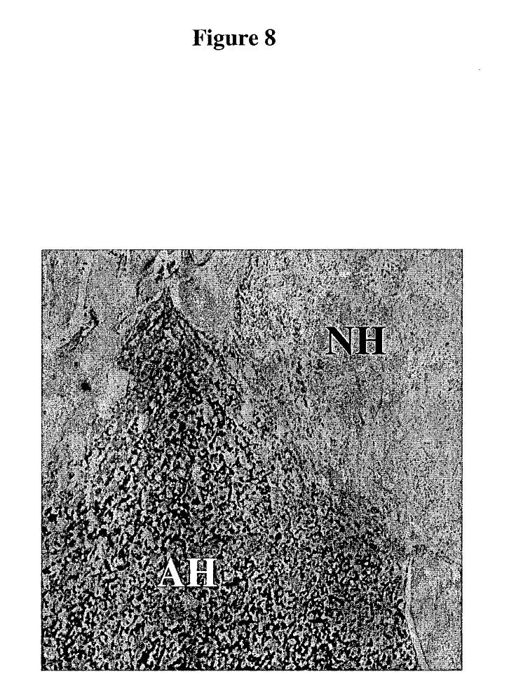

The expression and cellular localisation of human PDE11A protein was examined in human tissue sections by immunohistochemistry using an affinity-purified, anti-human PDE11A polyclonal antiserum (EPH-3). The results are shown in FIGS. 8 and 9.

Discussion

PDE11 is expressed in the acidophils of the anterior pituitary, i.e., somatotrophs and lactotrophs, with most other cells types, including those of the posterior pituitary, appearing negative (compare “AH” (positive for PDE11) with “NH” (negative for PDE11) in FIG. 8 and compare the “Anterior” (positive for PDE11) and “Posterior” (negative for PDE11) immunostains in FIG. 9).

The presence in acidophils suggests a role in modulating secretion of prolactin and/or growth hormone, which are secreted from the lactotrophs and somatotrophs respectively. For example, NO has been demonstrated to inhibit prolactin secretion via elevation of cGMP (Duvilanski et al., 1995). Therefore, elevation of cGMP levels in lactotrophs via PDE inhibition, for example, may result in reduced secretion of prolactin. Prolactin, in addition to its well-established role in lactation, has a diverse range of activities, which include effects on luteal function, steroidogenesis, immuno-regulation, growth, testis development and spermatogenesis (Bole-Feysot et al., 1998). Mice that are null for the prolactin receptor exhibit infertility, lack normal mammary development and depressed maternal behavior (females), delayed fertility (males), reduced bone formation and a slight reduction in body weight (Kelly et al., 2001), and hence these effects could be manifest in response to reduced or ablated prolactin secretion.

Cyclic AMP elevation in somatotrophs, in response to growth hormone-releasing hormone (GHRH) stimulation, results in the release of growth hormone (GH) from secretory granules (Mayo et al., 1995), and hence PDE inhibition may enhance this release. GH is important for growth stimulation but also persists throughout life after cessation of skeletal growth, and hence is thought to be important for maintenance, i.e., GH has protein and osteoanabolic, lipolytic and antinatriuretic properties (Murray & Shalet, 2000). Therefore, GH could have utility in the treatment of frailty associated with ageing, osteoporosis, morbid obesity, cardiac failure, major thermal injury and various acute and chronic catabolic conditions.

In conclusion, PDE11A may have a physiological role to play in modulating prolactin and/or growth hormone, and hence a range of direct and/or indirect/endocrine effects, such lactation, fertility, steroidogenesis, immuno-regulation and growth.

Knockout (KO) Mouse Data

Mice deficient in PDE11 were generated through targeted mutation of the gene coding for PDE11 (see above). Knockout mice and genetically matched wild type controls for plasma hormone assays on a hybrid B6:129 strain were bred by mating heterozygous males and females under standard environmental and dietary conditions in a positive pressure isolator. Light/dark cycle was controlled artificially. Animals were killed with carbon dioxide at a standard time into the light period and blood taken immediately from the abdominal vena cava to yield plasma.

Prolactin Assay

Determined in plasma from n=6 male WT mice and n=6 male KO mice. Prolactin was assayed using a commercial test kit (Rat Prolactin RPA553, Amersham Life Sciences, UK) in accordance with the manufacturer's recommendations.

Data Analysis

Data were analysed using an analysis of covariance (ANOCOVA) with age as a covariate. This tests for a linear relationship between age and response. “Parents” was included as a factor in the model. This is used to identify differences between parents after possible age differences are removed (N. B. Any detected difference attributed to age could, in fact, be a difference due to parents that happens to correspond to differences in age). Differences between the two genotypes were assessed after allowing for possible differences due to age and parents. The genotype means quoted also are estimated after these age and parent differences are removed.

Results

Prolactin (ng/ml)

| | WT | PDE11 KO |
|---|---|---|
| | 2.0 | 1.9 |
| | 2.7 | 0.4 |
| | 1.8 | 1.2 |
| | 0.6 | 0.5 |
| | 1.00 | 0.9 |
| | 0.4 | 0 |
| Mean | 1.42 | 0.82 |

Mice that are null for the prolactin receptor exhibit infertility, lack normal mammary development and depressed maternal behavior (females), delayed fertility (males), reduced bone formation and a slight reduction in body weight.

Conclusion

Plasma hormone assays (n=6 male mice) shows a clear relationship between age and prolactin level, and a distinct trend to reduced prolactin in the PDE11 KO mice (p=0.12).

G. Therapeutic Applications

Agents identified as modulating PDE11A activity can be used for therapeutic purposes. Preferably, the agent is selective for PDE11A with respect to at least one other PDE (e.g. Cialis (IC351), E4021, and UK-235,187 are selective for PDE11A, as compared to PDEs 7-10), more preferably, the agent is selective for PDE11A with respect to at least one of PDE3, PDE4, PDE5, and/or PDE6 (e.g. Cialis (IC351) is selective for PDE11A as compared to PDE6).

Modulation of Spermatozoa Capacitation

An exemplary therapeutic application of an agent of the present invention would be administering the agent to modulate spermatozoa capacitation for use as either a male in vivo contraceptive/male ex vivo pro-fertility agent (by decreasing PDE11A activity and increasing spermatozoa capacitation) or to increase male in vivo fertility (by stimulating PDE11A activity and decreasing spermatozoa capacitation). Agents that modulate PDE11A activity may be administered by any appropriate route. For example, administration may be parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

In addition to spermatogenesis, cAMP has also been reported to be an important messenger in the maintenance of motility in mature sperm (Chaudhry et al., Cell Motil. Cytoskeleton 32: 65-79, 1995). Furthermore, it also has a key role in eliciting the signaling events leading to capacitation and the acrosome reaction in sperm (Duncan & Fraser, J. Reprod. Fertil. 97: 287-99, 1993; Aitken et al., J. Cell Sci. 111: 645-656, 1998; Adeoya-Osiguwa & Fraser, Mol. Reprod. Dev. 57: 384-92, 2000); the latter being important to sperm activation and fertility. Consequently, perturbation of cAMP levels in spermatocytes, spermatids and spermatozoa via PDE11A inhibition, for example, can suppress spermatogenesis, but enhance sperm motility and/or sperm activation upon ejaculation.

Modulation of Prolactin and/or Growth Hormone Levels

PDE11A is expressed in the acidophils of the anterior pituitary, i.e., somatotrophs and lactotrophs. The presence of PDE11A in acidophils indicates that PDE11A plays a role in modulating the secretion of prolactin and/or growth hormone, which are secreted from the lactotrophs and somatotrophs respectively. For example, NO has been demonstrated to inhibit prolactin secretion via elevation of cGMP (Duvilanski et al., Proc. Natl. Acad. Sci. USA 92: 170-74, 1995). Therefore, the elevation of cGMP levels in lactotrophs produced via PDE11A inhibition, for example, can be useful to reduce secretion of prolactin for treating disorders related to hyperprolactinemia. Prolactin, in addition to its well-established role in lactation, has a diverse range of activities, which include effects on luteal function, steroidogenesis, immuno-regulation, growth, testis development and spermatogenesis (Bole-Feysot et al., Endocr. Rev. 19: 225-68, 1998). Mice that are null for the prolactin receptor exhibit infertility, lack normal mammary development and depressed maternal behavior (females), delayed fertility (males), reduced bone formation and a slight reduction in body weight (Kelly et al., Front. Neuroendocrinol. 22: 140-45, 2001). These effects could be manifest in response to reduced or ablated prolactin secretion. Accordingly, a PDE11A agonist can be used to decrease the levels of cAMP and cGMP in anterior pituitary lactotrophs and increase prolactin secretion and, conversely, a PDE11A antagonist can be used to decrease prolactin secretion.

Cyclic AMP elevation in somatotrophs, in response to growth hormone-releasing hormone (GHRH) stimulation, results in the release of growth hormone (GH) from secretory granules (Mayo et al., Recent Prog. Horm. Res. 50: 35-73, 1995), and hence PDE11A inhibition in anterior pituitary somatotrophs, for example, can enhance this release.

Exemplary therapeutic applications of modulating prolactin and/or growth hormone levels via PDE11 modulation would include:

(1) Inhibition of PDE11 and Hence Reduced Prolatin (PRL) Levels:

Electrolyte Balance

PRL reduces renal Na⁺ and K⁺ excretion, hence reduced PRL will increase/normalise excretion. Hence utility of an agent that reduces PRL levels as an anti-hypertensive.

Growth & Development

PRL stimulates proliferation of epithelial and smooth muscle cells, hence reduced PRL will decrease/normalise cell proliferation. Hence utility of an agent that reduces PRL levels in treating many cell proliferation disorders, e.g., restenosis and benign prostatic hyperplasia (BPH).

Reduced PRL will also have utility in preventing/reducing PRL-sensitive tumour growth, e.g., colorectal tumours and breast cancers (PRL is known to promote breast growth and lactation).

Reduced PRL also associated with longevity, hence utility of an agent that reduces PRL levels in ageing.

Endocrinology/metabolism

Reduced PRL will decrease phospholipid synthesis and increase carbohydrate metabolism, which supports evidence for utility of an agent that reduces PRL levels in reducing growth/weight gain (anti-obesity).

Brain & Behaviour

Reduced PRL will decrease maternal behaviour.

Reproduction

Decreased PRL will reduce mammary gland growth and lactation.

Reduced PRL will reduce fertility, e.g., ovulation and implantation. Hence utility of an agent that reduces PRL levels as a female contraceptive.

Reduced PRL will reduce fertility, i.e., spermatocyte to spermatid conversion. Hence utility of an agent that reduces PRL levels as a male contraceptive (in addition to capacitation effects).

Sexual Behaviour

Decreased PRL (via PDE11 inhibition, for example) will:

increase sexual desire (increase libido);

decrease sexual arousal; and

decrease/normalise orgasm.

Decreasing PDE11 activity (e.g. by providing a PDE11 inhibitor or antagonist to a female) would likely increase sexual desire (increase libido) in said female. Thus, PDE11 inhibition has a utility in preventing or treating female sexual dysfunction (FSD), e.g. hypoactive sexual desire disorder (HSDD).

(2) Enhancement of PDE11 Pathway and Hence Increased Prolactin Secretion:

Opposite of above (as set out under (1) above) plus:

Immunomodulation

PRL has been shown to enhance immune responses. Hence utility of an agent that increases PRL levels to enhance immune responses.

Sexual Behaviour

Increased PRL (via PDE11 stimulation, for example) will:

decrease sexual desire (decrease libido);

increase sexual arousal; and

increase/normalise orgasm.

Increasing PDE11 activity (e.g. by providing a PDE11 stimulator, activator, enhancer or agonist to a female) would likely increase sexual arousal and increase/normalise orgasm in said female. Thus, PDE11 stimulation has a utility in preventing or treating female sexual dysfunction (FSD), e.g. female sexual arousal disorder (FSAD), female orgasmic disorder (FOD) or sexual pain disorders.

(3) Inhibition of PDE11 and Hence Increased Growth Hormone (GH) Levels:

PDE11 inhibition may enhance growth hormone (GH) release. GH is important for growth stimulation, specifically bone growth, but also persists throughout life after cessation of skeletal growth, and hence is thought to be important for maintenance, i.e. GH has protein and osteoanabolic, lipolytic and antinatriuretic properties (Murray & Shalet, Expert Opin. Pharmacother. 1: 975-90, 2000). Therefore, an agent that increases GH levels could have utility in the treatment of frailty associated with ageing, osteoporosis, morbid obesity, cardiac failure, major thermal injury and various acute and chronic catabolic conditions.

When administering therapeutic formulations comprising an agent that 20 modulates PDE11A activity, the formulations may be in the form of liquid solutions or suspensions, in the form of tablets or capsules, or in the form of powders, nasal drops, or aerosols. Methods well known in the art for making formulations are found, for example, in Remington's Pharmaceutical Sciences (ed. Gennaro, Mack Publishing Co., Easton, Pa., USA, 18^{th} ed., 1990).

References

1) Duvilanski B H, Zambruno C, Seilicovich A, Pisera D, Lasaga M, Diaz M C, Belova N, Rettori V & McCann S M. Role of nitric oxide in control of prolactin release by the adenohypophysis. Proc. Natl. Acad. Sci. USA (1995); 92(1): 170-4.

2) Bole-Feysot C, Goffin V, Edery M, Binart N & Kelly P A. Prolactin (PRL) and its receptor: actions, signal transduction pathways and phenotypes observed in PRL receptor knockout mice. Endocr. Rev. (1998); 19(3): 225-68.

3) Kelly P A, Binart N, Lucas B, Bouchard B & Goffin V. Implications of multiple phenotypes observed in prolactin receptor knockout mice. Front. Neuroendocrinol. (2001); 22(2): 140-5.

4) Mayo K E, Godfrey P A, Suhr S T, Kulik D J & Rahal J O. Growth hormone-releasing hormone: synthesis and signaling. Recent Prog. Horm. Res. (1995); 50: 35-73.

5) Murray R D & Shalet S M. Growth hormone: current and future therapeutic applications. Expert Opin. Pharmacother. (2000); 1(5): 975-90.

1. A method of modulating in vivo or ex vivo spermatozoa capacitation, said method comprising administering an agent that modulates PDE11A activity.

2. The method according to claim 1, wherein said agent reduces PDE11A activity and increases in vivo or ex vivo spermatozoa capacitation.

3. The method according to claim 1, wherein said agent increases PDE11A activity and decreases in vivo or ex vivo spermatozoa capacitation.

4. Use of an agent that modulates PDE11A activity in the manufacture of a medicament for modulating in vivo or ex vivo spermatozoa capacitation.

5. Use according to claim 4, wherein said agent reduces PDE11A activity and increases in vivo or ex vivo spermatozoa capacitation.

6. Use of an agent that increases PDE11A activity in the manufacture of a medicament for decreasing in vivo or ex vivo spermatozoa capacitation.

7. Use of an agent that reduces PDE11A activity as a male pro-fertility agent, wherein said male pro-fertility agent is used ex vivo.

8. Use of an agent that increases PDE11A activity as a male pro-fertility agent, wherein said male pro-fertility agent is used in vivo.

9. Use of an agent that increases spermatozoa capacitation as a male pro-fertility agent, wherein said male pro-fertility agent is used ex vivo.

10. Use of an agent that reduces spermatozoa capacitation as a male pro-fertility agent, wherein said male pro-fertility agent is used in vivo.

11. Use of an agent that reduces PDE11A activity as a male contraceptive agent, wherein said male contraceptive agent is used in vivo.

12. Use of an agent that increases PDE11A activity as a male contraceptive agent, wherein said male contraceptive agent is used ex vivo.

13. Use of an agent that increases spermatozoa capacitation as a male contraceptive agent, wherein said male contraceptive agent is used in vivo.

14. Use of an agent that reduces spermatozoa capacitation as a male contraceptive agent, wherein said male contraceptive agent is used ex vivo.

15. A method of preventing or treating female sexual dysfunction (FSD), said method comprising administering to a female mammal an agent that stimulates, activates, enhances or agonises PDE11A activity.

16. A method according to claim 15, wherein said female sexual dysfunction (FSD) is female sexual arousal disorder (FSAD), female orgasmic disorder (FOD) or a sexual pain disorder.

17. Use of an agent that stimulates, activates, enhances or agonises PDE11A activity in the manufacture of a medicament for the prevention or treatment of female sexual dysfunction (FSD).

18. Use according to claim 17, wherein said female sexual dysfunction (FSD) is female sexual arousal disorder (FSAD), female orgasmic disorder (FOD) or a sexual pain disorder.

19. A method of preventing or treating female sexual dysfunction (FSD), said method comprising administering to a female mammal an agent that inhibits, decreases, deactivates or antagonises PDE11A activity.

20. A method according to claim 19, wherein said female sexual dysfunction (FSD) is hypoactive sexual desire disorder (HSDD).

21. Use of an agent that inhibits, decreases, deactivates or antagonises PDE11A activity in the manufacture of a medicament for the prevention or treatment of female sexual dysfunction (FSD).

22. Use according to claim 21, wherein said female sexual dysfunction (FSD) is hypoactive sexual desire disorder (HSDD).

23. The method according to any one of claims 1, 2, 19 or 20 or the use according to any one of claims 4, 5, 7, 9, 11, 13, 21 or 22, wherein said agent is Cialis (IC351), E4021 or UK-235,187.
